SEQUENCE LISTING (17 sequences)

SEQ ID NO: 1 - DNA, 1000 bp, Thraustochytrium sp. ONC-T18
  source 1..1000: mol_type = genomic DNA
aggatcaaagtcatactatgcgtacacgccgcgttcggaaaccctagctggttcaaccagttccctcttctgattccctcgctgggttctgcgggccacgctcaagccgtccgggacgtcatggacgtcgcgctgccctgcgtcgttcttctacgcgtacgcacaagaaggcgtcaccgccgcgcccgcgccgaagacctccctcccgatcgaaggtcctggttctcgggaggcgctgtgcgtggtatgtcgacgcgctcggctctgcgctggagagcgcaaggcggctttttgaccaggttgcctgcctcctaccacgtgcccgtagggagggggaatgtaccgcagtgcggtggtccgccaagcaagaaaccccgcagagaaggcgtaaagtggaagaaaaacagcgtcgtatgccgccgtcgtcgcaggtgctcgtcgtcgcctcgtcgatgggacccatcatgcgctgagagtctgctgcaaaagaggtagggactcgggaaggacctggttcgcgctggctggggaatcagtaatcgcatttcggacatggatgcggagacgctccccgatgacgatgctccaggaatcgcgcggcacgttttacggcggcgagagagaaagtctcagcttcttttcgagggtatactccgtggcgggttatcatcatgtgagagatgtatcgacgcgatggagaagcatcgggtcttcgtcgaacatccggggtcgccggttcgtcacgaagccagctcatgcctccacatgtctcacaagaccaccgaaaaactgtcgaacttgattctttaggtctctcggacgcaaataaaaagcatcggcgcgctcgcgttcaccagcaagcagacaaaaccaatatcagcctattggccgaccaaaaccaagcagcagttcctcaactcggctctgctcaactcagcaaactgccaacgcgtactagcagacaaggattcaccccagcttcggttgaaactacaagatc

SEQ ID NO: 2 - DNA, 1134 bp, Oblongichytrium sp.
  source 1..1134: mol_type = genomic DNA
atggccacctccatgaacgactccctgcccgagggcgccaccgccatgaacaccctgcagcgcctgctgtccttccagaacgagttccactccaaggaggtcctgacctggcaccgggaccacgccgagatccccatcatctgcctgtccctgtacctggtcatggtcttcgccggccccgacctgatgaagcaccgcgagcccttcaagctgaagcgcaccttcgccgcctggaacttcttcctgtccgtcttctccatcctgggcgcctaccacatggtccccctgctgctgggccgcctgtgggagcacggcttcaaggccaccgtctgctcccaccccgagtggtacttcaacggcccctccggcttctggctgtgcctgttcatctactccaagttcgccgagctgttcgacaccgccttcctggtcctgcgcaagcgcaacgtcatcttcctgcactggttccaccacgccaccgtcctgctgtactgctggcacgcctaccaccacgccatcggcgccggcgtctggttcgcctgcatgaactactgcgtccactccgtcatgtacttctactacttcatgaccaacgtcggcctgtatcgcgtcgtcgcccccttcgcccaggccatcaccaccgtccagatcctgcagatggtcggcggcatggccgtcctgctgcaggtcgcccacgcccgcctgaccgaggaccccaccgcctgcgaggtcgactccgccaactggaagctgggcctggccatgtacgcctcctacttcgtcctgttcgtcctgctgttcgtcaagaagtatctgtcccccgccccctcctccgcccagtccaagcgcacctccggcaacgacacctcctccgccaccggctccaccaccaagtccaactcctcctccaccaagggcgacgccgacgcctccgccggcgcccgccgccgcgcccgcgtctccgtcacctgcccccccggcctggacgacgtccccgtcttcggcgactccgcctccgtctccaagctggacgcctccggcttcttccactcccagcagaccgtcgaggaggcccgccgggcccaggacgacgccgaccgcgcccaggccatgcggaacaagaagaaggccctg

SEQ ID NO: 3 - DNA, 1158 bp, Ulkenia sp.
  source 1..1158: mol_type = genomic DNA
atgacccacaactcctccttcgccggcgacgccctgcacgccgacgcctccgtcaaccgcgtcgccggcctggccggcaccgtcgtcttcggcaccgtcctggcctacctgggcacccggcagcccaaggagtcccactccatgaagctgcgcgacatgcccgagggctataaggcccagaacgacctggagcagaaggccatcgacgcctacgagcgccggcaggagctgtccaccatcccctggctgttccagaacatccgctgggtcatgtccatctacatcggcggcatccacctgctggccatctatgccctgccccacctgctggactgcaagtgggagaccctgctgggcatgctggccttctacgtcctgggcggcttcggcatcaccggcggcgcccaccgcctgtggtcccaccgcacctacaaggccaacgccgtcttccgcttcgtcgtcatgatctgcaactccatcgccaaccagggcaccatctaccactggtcccgcgaccaccgcacccaccacaagtactccgagaccaaggccgacccccacaacgccctgcgcggcttcttcttcgcccacgtcggctggctgttcttcaagaaggacccccgggtcaagttcgccggcaaccacatccccatgaacgacctggccgccctgcccgaggtccagctgcagaagcgcctggacccctggtggaacctgttctggtgcttcggcgcccccaccctgtgcggccacttcctgtggggcgagaccgtcctgaagtccttcctgctgatgggcgtcctgcgctatgccctgtgcctgaacggcacctggctggtcaactccgccgcccacctgtacggcggccacccctatgaggacatcaaccccgccgagaaccccgtcgtcgccttcttctccatgggcgagggctggcacaactggcaccacgccttcccccacgactattccgcctccgagctgggcgtctcctcccagttcaaccccacccgcctggtcatcgacttctgcgccctgttcggcatcgtctgggaccgcaagaccgccaccgaccactgggagacccgcaagaagaagaagggctataagcacgtcgacctccagggcgcccccttcttccggcagcgcgtcgtctcc

SEQ ID NO: 4 - DNA, 1238 bp, Thraustochytrium sp. ONC-T18
  source 1..1238: mol_type = genomic DNA
atgtgcaaggtggagcccacgcggcacgagcagacggctgcggccaagccgcaagagcagcagcagcagcgtcagagtcccatcatccatggcaagcacaacccggacctgccgacgctcggcgagattcgcgccgtggtgccgaagcactgctttgagcgctcgctcgtgacgagctcgctttacctgggccgcgaccttctcatggcagccaccctcttcttcctcgccagacaattccttcccgtttacgacatggcctctcgggcgcgctcgcctggactgtctacgtctgtgtgcagggtaccgtcggtgccggcctttgggtgcttgggcatgaatgcggtcaccaggccttttccaactacaggatcgtcaacgacggcgtcggctttctggtccacacgagcctgttggtgccttatttcagctgggcgtacacgcacggcttgcaccatgcccgcgtcaaccacatgctcgacggcgagtcgcacacgccgaacctgaagaagaaggtggccgccaactttcaaaagttctgcgacatgatgggcgatgaggcgtttgccgttcttcacgtcttcgtctaccttctcctggcctggccgctttacatcatcaacgggagcggcgcgtccaagcgcaaccacgagggaaagcgctggtcgaaggatttctggaagcgccccaaccactttttgcccacctcggagctctttccggacaagatgcgcctcaaggccgccgtctccacgatcggcgtccttaccgtcattgccggcctctgctactggggctctatcgagggcgggcgcaccgtgctgctccagtactttctgccctacctggtggtcaatgcctatctcattgggtttacctggatgcaacacacccacccggacgtcccgcacctcggcgaggacgagtggtcctgggtcgcgggcactgtgctcaccgtcgatcgcccctacccgccttttatcgacgttctgacccaccgcatcgggtctacgcacgtggcgcatcacctcttctccaagatgccgtggtaccacgcgcgcgaggctacgacccacatccggaccctcctcgagcccaagggcgtctacaactatgacccgatgcccttttacaaggccttgtttcacactgctaagtactgccactacatggagggcgtcgacggtattcagttcttcaaacatgccgctgcccagcccaaggccaaggagctctaa

SEQ ID NO: 5 - DNA, 1215 bp, Thraustochytrium sp.
  source 1..1215: mol_type = genomic DNA
atgtgcaagaacgaggcccagtccaagtccgccgccctgcgcggcgcccccccccagcagcaggaccagcccctgccctccatcaaggacatccgcgccgccgtccccgcccactgcttccagcgctccgccctgcggtcctccctgttcgtcgtccgggacgccgccctggccgccctggtcggctgggccgcctacaagaccctgcccaccgacctgggcaaccccctggccctggccggctggctggcctacgccctggtccagggcaccgtcctgaccggcctgtgggtcatcggccacgagtgcggccaccaggccttctccgagtccgccctggtcaacgactccttcggcttcgtcatccactccgccctgctggtcccctacttctcctgggcccgcacccacgccgtccaccacgcccgctgcaaccacctgctggacggcgagacccacgtccccgacctgaagcgcaaggtccacggcatgtacgccaagatcctggacgtcgtcggcgaggacgccttcgtcctgctgcagatcgtcctgcacctgctgttcggctggatcatgtatctggtcatgcacgccaccggctcccgccgctcccccgtcaccaaggagcggtacaagcgcaagcccaaccacttcgtccccctggcctccaacgagctgttccccgccaagctgcgcttcaaggtcctgctgtccaccgtcggcgtcctgggcatgatcggcgccctgtgctatgccggctccctgtatggcggcaagatcgtctccctgctgtacgtcggcccctacctggtcgtcaacgcctggctggtcacctatacctggctgcagcacaccgaccccgaggtcccccactacggcgaggccgagtggacctggctgaagggcgccctgtccaccatcgaccgcccctacccctggatcgtcgacgagctgcaccaccacatcggcaccacccacgtcacccaccacgtcttccacgagctgccccactaccacgcccaggaggccaccgccgccctgaaggccgtcctgggcccccactatcgctacgaccccacccccatcgtcaaggccatgtggaagaccgccgagacctgccactacgtcgaggacgtcaagggcgtccagtacctgcggtccatcgtcaccgagcgccgccaggccgcccaggccgccgccaaggccaaggccctg

SEQ ID NO: 6 - DNA, 2590 bp, Shewanella sp.
  source 1..2590: mol_type = genomic DNA
atgtcgtcgcagcactcgaccgcctcgcagtcgaacaagatcgccatcgtcggcctcgccaaccagtacccggacgccgacaccccgaaggacttctggcagaacctcctcgccaagaaggactcgcgcaccaccctctcgccggagaagctcggcgccaacccggacgcctaccagggcatccagggcgagtcggaccgcttctactgcgacaagggcggctacatccagaacttctcgttcgactcgaacggctaccgcctcccggccgagaccttcgagggcctcgacgagtcgttcctctgggccctcgacacctcgcgcaaggccctcgccgacgccggcatcccgctcgacgacgccgtcctcgagcgcaccggcgtcatcatgggcgccctctcgttcccgaccacccgctcgaacgacctcttcctcccgatctaccactcggtcgtcgagaaggccctccagaccaagctcggcaacgagcacttctcgctcatcccgtcgaacgccaacatcacctcgctcaacccggccaacggctcggccgcccacaacgcctcgaagctcgtcgccgacgccctcggcctcggctcggcccagctctcgctcgacgccgcctgcgcctcgtcggtctactcgctcaagctcgcctgcgactacctctcgaccggcaaggccgacatgatgctcgccggcgccgtctcgggcgccgacccgttcttcatcaacatgggcttctcgatcttccacgcctacccggaccacggcgtctcggtcccgttcgacaccaactcgaagggcctcttcgccggcgagggcgccggcgtcctcgtcctcaagcgcctcgaggacgccgagcgcgacggcgacaacatctacgccgtcgtctcgggcatcggcctctcgaacgacggccgcggccagttcgtcctctcgccgaactcgaagggccaggtccaggccttcgagcgcgcctacgaggccaccaacctctcgccggagtcgatcgaggtcatcgagtgccacgccaccggcaccccgctcggcgacaaggtcgagatgacctcgatggagcgcttcttcgccgacaagctcaacggctcgcaggccccgctcatcggctcggccaagtcgaacctcggccacctcctcaccgccgccggcatgccgggcatcatgaagatgatcttcgccatgaaggagggcgtcctcccgccgtcgatcaacctctcgaccccgctctcgtcgccggagggcctcttcggctcgcacaccctcccgacccaggtccaggcctggccggacaaggccggcaacaccgagcgctgcgccggcgtctcggtcttcggcttcggcggctgcaacgcccacctcctcctcgaggcccactcggccaactcggccgccaactcgccggccgccaactcgccggccaagccggtcacctcggccccgctcaagatcaccggcctcgtctcgcacttcggctcgctcaagaccgtcaacgccctccacaacgccatcaccaccggcaccgacgccttcgtcgccctcccgaagaagcgctggaagggcctcgaccagcacccggagctcctctcgcagttcggcctcgaggccatcccgcacggcgcctacatcgaccagttcgagctcgacttcctccgcttcaaggtcccgccgaacgaggacgaccgcctcatctcgcagcagctcctcctcatgaaggtcgccgacgaggccatccgcgacgccaagctcgaggccggccagaaggtcgccgtcctcgtcgccatggagaccgagctcgagatgcaccagttccgtggccgcgtcaacctccacacccagctcgccgactcgctccagaacatgggcgtccagctcaccgactcggagtaccaggccctcgaggccatcgccatggactcggtcctcgacgccgccaagctcaaccagtacacctcgttcatcggcaacatcatggcctcgcgcatcgcctcgctctgggacttcaacggcccggccttcaccatctcggccgccgagcagtcggtcgcccgctgcatcgacgtcgcccagaacctcatgtcgcaggagtcgctcgacgccgtcgtcatcgccgccgtcgacctctcgggctcggccgagcagatcatcctcaagaactcggtcaccccggtcgccctccacgcccaggactcgggctggaacgtcggcgagggcgccggcgccatcgtcctcgtcgacaccgacaacgcccagacccagaactcgtacggcgagatcaccgccctcaacttcggctcggtcgcccagtcgaacatcatctcggaccgcctcctcaccaccgccggcatcgccgccaacaacgtctacctcctcgagctcaaccaggccccggagtcggtcgacgccgtccagttcccgctcccgtcggccacccacatccaggccaaccagcgcctcggccactgctacgccgcctcgggcatggcctcgatcctccacggcctcctctcgctcaacgccatcccgaagcagaccaccatcccgtcgctcaacacctcggtcaccgccgccg

SEQ ID NO: 7 - DNA, 1308 bp, Oblongichytrium sp.
  source 1..1308: mol_type = genomic DNA
atgtgcaaccccaagtccctgtccgccccctccggcgtcgaggcccacgtcgtccaggagcacgtccccgtcggcgagcacgacaagtggctggagaccctggacttcgacgccttcaaggaggacatgcagcgcctgggcaagtccctggccgacggccagggccagcaggacgtcgaccacctgcagaagttcgtctggtggaaccgcatcgtcaccctgtgcggcatcctgaccatggcctgcttccccaaccccttcaccgtcttctgcctgtccctgggcaccttctcccgctggaccatcatcgcccaccacgtctgccacggcggcttcgacaaggccgacaagtccaagcgctataaccgcttcaccttcggcgtcggctccctgtaccgccgcatggtcgactggttcgactggatgctgcccgaggcctggaacgtcgagcacaaccagatgcaccactaccacctgaacgaggccaaggaccccgacctggtcgagctgaacctgtcccccctgcgcgagaacgacgccgtccccatgcccgtcaagtacatggtcgtcttcttcttcatgtgcacctggaagtggttctactacgccccctccaccttcatccagctggcccaggcccgcctgcgccgccagggcgtctccaccgagggcatgccccgggcccacctgaccatcgcctccatcttcgaggcccccgacttcgtctccatcaaggagctgttcctgcgggtcctgttcccctatttcgcctaccgcttcctgctggtccccctgcccgtcctggccctgtggggccccaagctgttctcctacgccatcatcaacctggtcctggccgacatcgtcaccaacatccactccttcatcgccgtcgtcaccaaccacgccggcgacgacctgtatgtcttcgagaagcactgcaagccccggggcgcccacttctacctgcggcaggtcatctcctccgtcgacttcacctgcggcaacgacctgatggacttcatgcacggcttcctgaactaccagatcgagcaccacctgtggcccgacctgtccatgctgtcctaccagcgcgcccagcccctggtcaagcagctgtgcgccaagcacggcgtcccctacgtccaggagtccgtctggatccgcctgaagaagaccatcgacatcatggtcggcaagacctcccaccggaagttcccctcccagtacgagcccaacgactatgaccacctgaagaccgccgccgccaccaccaccgccatggaggagcccgaggccgaggccaag

SEQ ID NO: 8 - DNA, 1371 bp, Botryochytrium sp.
  source 1..1371: mol_type = genomic DNA
atgggccgcggcggccagaagacggagggcctttcggcccagccccagcaggagcgcgagcagcttctcaagggcaagtgggagtccgtggtccgcatcgacggcgtcgagtacgacgtgacggactacatgcgcaagcaccccggcggctccgtcatcaagtacggccttgccaacaccggcgccgacgccacgcacctgttcaacgcgtttcacatgcgctcgaagaaggccaagatggtgctcaagtcgctgcccaagcgccagccccagctcgagatccagcccggccagcttcccgaggagcagacgaaggaggccgagatgcttcgcgacttcgagaagctcgagaacgagcttcgcgccgagggctacttcgagccctcgttctggcaccgcctctatcgcttcaccgagcttgccgtcatgttttcccttgggctctactgcttctcgctgcgcacgccgctttcgatcgccgccggcgtgttcctccacggcctctttggcgccttctgcggctgggcgcagcacgagggcggccacgggtccctttaccactccctttggtggggcaagcgcgcccaggccatgatgattggtttcggcctcgggacgtccggcgacatgtggaacatgatgcacaacaagcaccacgcggccacgcagaaggtgaaccacgacctcgacatcgacacgacgccgcttgtcgcgttcttcaacacggcgtttgagaagaaccgctatcgcggcttcgccaagtggtgggtccgctttcaggccctcaccttccttcccatcacctcgggctgcttcgtgatgtggttctggctcctcttcctgcacccgcgccgcgtggtccagaagggcaacgtcgaggaggggttctggatgctctcctcgcacatcgtccgcacgtacctgtttcagctctgcaccggctgggagtcccttgccgcctgctacctggtcggctactggggcgccatgtgggtgtccggcgtgtacctgtttggccacttctccctttcgcacacccacctggacatcgtcgacgcggacgtgcacaagaactgggtccgctacgccgtggaccacaccgtcgacatttcgccccaaaacccgctggtgtcctggatcatggggtacctcaacctccaggtgctgcaccacctctggccccagatgccccagtaccaccagcccgccgtctccaagcgcgtcgcggccttctgcaagaagcacggcctgaactatcgcgtcgtgtcgtacttcgaggcgtggaagctcatgttctcgaacctctccaacgtctccgaccactacatgaagaacggcttcgagcgccccgccaagaagacgaaggcccag

SEQ ID NO: 9 - DNA, 1089 bp, Pavlova pinguis
  source 1..1089: mol_type = genomic DNA
atgtgcccgccggcgacccacgacgcgaccccgatcaaggacggcgcgaaccgcgccgagatcgtcgccgagtccaagctgacccttcaggacattcgcaaggcgattccgcaggagtgcttcgagaagaacacggcgcgctccatgctgtaccttgtgcgcgacctggccatttgcgcgaccgcgcccctggtgtacccctacgtcgcggcctcgggcaacccgctcgcctacctcgcctattggaacttctacgggttcttcatgtggtgcctcttcgtcgtcggccacgactgcggccacaccacgttctccccgaacaagaccctgaacgatatttgcggccacatcgcccacgcgccgctcatggtgccctactatccctgggccatgtcgcaccgccgccaccacatgtaccacaaccaccagaagaaggacgcgtcccacccctggttctcgaagtcctccctcaagaagcttccggccttcacccgcaacttcctgaagtccccgcttgcgcccttccttgcctaccccatctacctcttcgagggctccttcgacgggtcccacgtcttcccgctttcgaagctctacaagggctcccagatgcgcgcccgcgtggagtgcgcgatttccgccgtgacggtcttcgcctttggcaccgccgcgtacatgttttgcggcgacgcccgcacccttgccctcgcctatggcggctgctacgcctgcttctccttctggctgttcatggtcacctacctccagcaccacgaccacggcaccctcgtgtacgacgactcggactggacctacctgaagggtgctctggagaccgtcgaccgcaagtacgggtttggcctcgacaacctgcaccacaacatctcggacggccacgtcgtccaccacctgttcttcacccaggtgccccactaccacctcacgaaggcgaccgagcaggtcgcgccgcttctccgcaaggccggcgtctacaagcgcgtcgaccacgacaacttcctcaaggacttctggcgcaccttctttacctgcaacttcaccggctggaagtgggccaacgggaaggacaac

SEQ ID NO: 10 - DNA, 1323 bp, Thraustochytrium sp.
  source 1..1323: mol_type = genomic DNA
atgggccgcggtggcgagggcgaggccccgaagcgcgccgagctccacggcgccacctctacgggccgcaaggtcgtgctcatcgagggccagatgtacgacgtcacgaactttcgccacccgggcggctccatcatcaagtttctgagcacggacggcagcgaggtcgtcgacgccacggaggcctaccgcgagttccactgccgctcctcgaccgcggacaagtacctcaaggcgctcccgaaggtcgacggcccgatcaagatgaagtttgacgcgaaggagcaggcccgccgggatgcgatcacgcgcgactacgccatcctgcgggagcagctcgtcaaggaggggttctttaagccggtcccgctccacgtcctctaccgctgcgtcgagatcctcgccatgtttgccctgtcgttctacctcttctcgttcaagaacaacatgctcgccatcgccgcggccgtcctcgtgggcgggatcgtccagggccgctgcggctggctcatgcacgaggccggccactacagcatgacgggctacatcccgctcgacctccgcctgcaagagctcatctatggggtgggctgcgggatgtcgggcgcctggtggcgcaaccagcacaacaagcaccatgccacgccgcagaagctcaagcacgacgtggacctggatacgctcccgctcgtcgcgtttaacgagaagatcgccgcccgcgtcaagccgggctccttccaggccaagtggctctcggcccaggcctacatcttcgcgccgatctcctgcctccttgtcggcctcttctggaccctctttctccacccgcgccacatgatccgcaccaagcgccgggccgagtttgtctggatcgtgacgcggtatctcggctggtttggcctgctccactcgttcggctactcgttcggcgacgccttcaagctctacctcgtcacctttggcgtcgggtgcacctacatcttcaccaacttcgccgtgtcgcacacgcatctccccgtcaccaacccggacgagtttctgcactgggtcgagtacgccgccctgcacaccacgaacgtctcgaacgactcgtggtttgtcacctggtggatgtcgtacctgaactttcagatcgagcaccacctcttcccgtgctgcccccagctccaccatccgaagattgccccgcgcgtccgccagctctttgagaagcacggcatggtctacgacgagcgcccgtacgtccaggccctcaaggacacctttaacaacctccacagcgtcggcaacgccgtcaactcctcgaagaagaccgccgaattc

SEQ ID NO: 11 - DNA, 2157 bp, synthetic construct
  source 1..2157: mol_type = other DNA
aggatcaaagtcatactatgcgtacacgccgcgttcggaaaccctagctggttcaaccagttccctcttctgattccctcgctgggttctgcgggccacgctcaagccgtccgggacgtcatggacgtcgcgctgccctgcgtcgttcttctacgcgtacgcacaagaaggcgtcaccgccgcgcccgcgccgaagacctccctcccgatcgaaggtcctggttctcgggaggcgctgtgcgtggtatgtcgacgcgctcggctctgcgctggagagcgcaaggcggctttttgaccaggttgcctgcctcctaccacgtgcccgtagggagggggaatgtaccgcagtgcggtggtccgccaagcaagaaaccccgcagagaaggcgtaaagtggaagaaaaacagcgtcgtatgccgccgtcgtcgcaggtgctcgtcgtcgcctcgtcgatgggacccatcatgcgctgagagtctgctgcaaaagaggtagggactcgggaaggacctggttcgcgctggctggggaatcagtaatcgcatttcggacatggatgcggagacgctccccgatgacgatgctccaggaatcgcgcggcacgttttacggcggcgagagagaaagtctcagcttcttttcgagggtatactccgtggcgggttatcatcatgtgagagatgtatcgacgcgatggagaagcatcgggtcttcgtcgaacatccggggtcgccggttcgtcacgaagccagctcatgcctccacatgtctcacaagaccaccgaaaaactgtcgaacttgattctttaggtctctcggacgcaaataaaaagcatcggcgcgctcgcgttcaccagcaagcagacaaaaccaatatcagcctattggccgaccaaaaccaagcagcagttcctcaactcggctctgctcaactcagcaaactgccaacgcgtactagcagacaaggattcaccccagcttcggttgaaactacaagatcggtacctcgcgaatgcatctagaatggccacctccatgaacgactccctgcccgagggcgccaccgccatgaacaccctgcagcgcctgctgtccttccagaacgagttccactccaaggaggtcctgacctggcaccgggaccacgccgagatccccatcatctgcctgtccctgtacctggtcatggtcttcgccggccccgacctgatgaagcaccgcgagcccttcaagctgaagcgcaccttcgccgcctggaacttcttcctgtccgtcttctccatcctgggcgcctaccacatggtccccctgctgctgggccgcctgtgggagcacggcttcaaggccaccgtctgctcccaccccgagtggtacttcaacggcccctccggcttctggctgtgcctgttcatctactccaagttcgccgagctgttcgacaccgccttcctggtcctgcgcaagcgcaacgtcatcttcctgcactggttccaccacgccaccgtcctgctgtactgctggcacgcctaccaccacgccatcggcgccggcgtctggttcgcctgcatgaactactgcgtccactccgtcatgtacttctactacttcatgaccaacgtcggcctgtatcgcgtcgtcgcccccttcgcccaggccatcaccaccgtccagatcctgcagatggtcggcggcatggccgtcctgctgcaggtcgcccacgcccgcctgaccgaggaccccaccgcctgcgaggtcgactccgccaactggaagctgggcctggccatgtacgcctcctacttcgtcctgttcgtcctgctgttcgtcaagaagtatctgtcccccgccccctcctccgcccagtccaagcgcacctccggcaacgacacctcctccgccaccggctccaccaccaagtccaactcctcctccaccaagggcgacgccgacgcctccgccggcgcccgccgccgcgcccgcgtctccgtcacctgcccccccggcctggacgacgtccccgtcttcggcgactccgcctccgtctccaagctggacgcctccggcttcttccactcccagcagaccgtcgaggaggcccgccgggcccaggacgacgccgaccgcgcccaggccatgcggaacaagaagaaggccctg

SEQ ID NO: 12 - DNA, 23 bp, synthetic construct
  source 1..23: mol_type = other DNA
ggtacctcgcgaatgcatctaga

SEQ ID NO: 13 - DNA, 2394 bp, synthetic construct
  source 1..2394: mol_type = other DNA
aggatcaaagtcatactatgcgtacacgccgcgttcggaaaccctagctggttcaaccagttccctcttctgattccctcgctgggttctgcgggccacgctcaagccgtccgggacgtcatggacgtcgcgctgccctgcgtcgttcttctacgcgtacgcacaagaaggcgtcaccgccgcgcccgcgccgaagacctccctcccgatcgaaggtcctggttctcgggaggcgctgtgcgtggtatgtcgacgcgctcggctctgcgctggagagcgcaaggcggctttttgaccaggttgcctgcctcctaccacgtgcccgtagggagggggaatgtaccgcagtgcggtggtccgccaagcaagaaaccccgcagagaaggcgtaaagtggaagaaaaacagcgtcgtatgccgccgtcgtcgcaggtgctcgtcgtcgcctcgtcgatgggacccatcatgcgctgagagtctgctgcaaaagaggtagggactcgggaaggacctggttcgcgctggctggggaatcagtaatcgcatttcggacatggatgcggagacgctccccgatgacgatgctccaggaatcgcgcggcacgttttacggcggcgagagagaaagtctcagcttcttttcgagggtatactccgtggcgggttatcatcatgtgagagatgtatcgacgcgatggagaagcatcgggtcttcgtcgaacatccggggtcgccggttcgtcacgaagccagctcatgcctccacatgtctcacaagaccaccgaaaaactgtcgaacttgattctttaggtctctcggacgcaaataaaaagcatcggcgcgctcgcgttcaccagcaagcagacaaaaccaatatcagcctattggccgaccaaaaccaagcagcagttcctcaactcggctctgctcaactcagcaaactgccaacgcgtactagcagacaaggattcaccccagcttcggttgaaactacaagatcggtacctcgcgaatgcatctagaatgggccgcggcggccagaagacggagggcctttcggcccagccccagcaggagcgcgagcagcttctcaagggcaagtgggagtccgtggtccgcatcgacggcgtcgagtacgacgtgacggactacatgcgcaagcaccccggcggctccgtcatcaagtacggccttgccaacaccggcgccgacgccacgcacctgttcaacgcgtttcacatgcgctcgaagaaggccaagatggtgctcaagtcgctgcccaagcgccagccccagctcgagatccagcccggccagcttcccgaggagcagacgaaggaggccgagatgcttcgcgacttcgagaagctcgagaacgagcttcgcgccgagggctacttcgagccctcgttctggcaccgcctctatcgcttcaccgagcttgccgtcatgttttcccttgggctctactgcttctcgctgcgcacgccgctttcgatcgccgccggcgtgttcctccacggcctctttggcgccttctgcggctgggcgcagcacgagggcggccacgggtccctttaccactccctttggtggggcaagcgcgcccaggccatgatgattggtttcggcctcgggacgtccggcgacatgtggaacatgatgcacaacaagcaccacgcggccacgcagaaggtgaaccacgacctcgacatcgacacgacgccgcttgtcgcgttcttcaacacggcgtttgagaagaaccgctatcgcggcttcgccaagtggtgggtccgctttcaggccctcaccttccttcccatcacctcgggctgcttcgtgatgtggttctggctcctcttcctgcacccgcgccgcgtggtccagaagggcaacgtcgaggaggggttctggatgctctcctcgcacatcgtccgcacgtacctgtttcagctctgcaccggctgggagtcccttgccgcctgctacctggtcggctactggggcgccatgtgggtgtccggcgtgtacctgtttggccacttctccctttcgcacacccacctggacatcgtcgacgcggacgtgcacaagaactgggtccgctacgccgtggaccacaccgtcgacatttcgccccaaaacccgctggtgtcctggatcatggggtacctcaacctccaggtgctgcaccacctctggccccagatgccccagtaccaccagcccgccgtctccaagcgcgtcgcggccttctgcaagaagcacggcctgaactatcgcgtcgtgtcgtacttcgaggcgtggaagctcatgttctcgaacctctccaacgtctccgaccactacatgaagaacggcttcgagcgccccgccaagaagacgaaggcccag

SEQ ID NO: 14 - DNA, 23 bp, synthetic construct
  source 1..23: mol_type = other DNA
ggtacctcgcgaatgcatctaga

SEQ ID NO: 15 - DNA, 2332 bp, synthetic construct
  source 1..2332: mol_type = other DNA
aggatcaaagtcatactatgcgtacacgccgcgttcggaaaccctagctggttcaaccagttccctcttctgattccctcgctgggttctgcgggccacgctcaagccgtccgggacgtcatggacgtcgcgctgccctgcgtcgttcttctacgcgtacgcacaagaaggcgtcaccgccgcgcccgcgccgaagacctccctcccgatcgaaggtcctggttctcgggaggcgctgtgcgtggtatgtcgacgcgctcggctctgcgctggagagcgcaaggcggctttttgaccaggttgcctgcctcctaccacgtgcccgtagggagggggaatgtaccgcagtgcggtggtccgccaagcaagaaaccccgcagagaaggcgtaaagtggaagaaaaacagcgtcgtatgccgccgtcgtcgcaggtgctcgtcgtcgcctcgtcgatgggacccatcatgcgctgagagtctgctgcaaaagaggtagggactcgggaaggacctggttcgcgctggctggggaatcagtaatcgcatttcggacatggatgcggagacgctccccgatgacgatgctccaggaatcgcgcggcacgttttacggcggcgagagagaaagtctcagcttcttttcgagggtatactccgtggcgggttatcatcatgtgagagatgtatcgacgcgatggagaagcatcgggtcttcgtcgaacatccggggtcgccggttcgtcacgaagccagctcatgcctccacatgtctcacaagaccaccgaaaaactgtcgaacttgattctttaggtctctcggacgcaaataaaaagcatcggcgcgctcgcgttcaccagcaagcagacaaaaccaatatcagcctattggccgaccaaaaccaagcagcagttcctcaactcggctctgctcaactcagcaaactgccaacgcgtactagcagacaaggattcaccccagcttcggttgaaactacaagatcggtacctcgcgaatgcatctagaatgtgcaaccccaagtccctgtccgccccctccggcgtcgaggcccacgtcgtccaggagcacgtccccgtcggcgagcacgacaagtggctggagaccctggacttcgacgccttcaaggaggacatgcagcgcctgggcaagtccctggccgacggccagggccagcaggacgtcgaccacctgcagaagttcgtctggtggaaccgcatcgtcaccctgtgcggcatcctgaccatggcctgcttccccaaccccttcaccgtcttctgcctgtccctgggcaccttctcccgctggaccatcatcgcccaccacgtctgccacggcggcttcgacaaggccgacaagtccaagcgctataaccgcttcaccttcggcgtcggctccctgtaccgccgcatggtcgactggttcgactggatgctgcccgaggcctggaacgtcgagcacaaccagatgcaccactaccacctgaacgaggccaaggaccccgacctggtcgagctgaacctgtcccccctgcgcgagaacgacgccgtccccatgcccgtcaagtacatggtcgtcttcttcttcatgtgcacctggaagtggttctactacgccccctccaccttcatccagctggcccaggcccgcctgcgccgccagggcgtctccaccgagggcatgccccgggcccacctgaccatcgcctccatcttcgaggcccccgacttcgtctccatcaaggagctgttcctgcgggtcctgttcccctatttcgcctaccgcttcctgctggtccccctgcccgtcctggccctgtggggccccaagctgttctcctacgccatcatcaacctggtcctggccgacatcgtcaccaacatccactccttcatcgccgtcgtcaccaaccacgccggcgacgacctgtatgtcttcgagaagcactgcaagccccggggcgcccacttctacctgcggcaggtcatctcctccgtcgacttcacctgcggcaacgacctgatggacttcatgcacggcttcctgaactaccagatcgagcaccacctgtggcccgacctgtccatgctgtcctaccagcgcgcccagcccctggtcaagcagctgtgcgccaagcacggcgtcccctacgtccaggagtccgtctggatccgcctgaagaagaccatcgacatcatggtcggcaagacctcccaccggaagttcccctcccagtacgagcccaacgactatgaccacctgaagaccgccgccgccaccaccaccgccatggaggagcccgaggccgaggccaagt

SEQ ID NO: 16 - DNA, 23 bp, synthetic construct
  source 1..23: mol_type = other DNA
ggtacctcgcgaatgcatctaga

SEQ ID NO: 17 - DNA, 828 bp, Thraustochytrium sp.
  source 1..828: mol_type = genomic DNA
atggaggtcgccgggcagcaatggcgccggttcgtggacgccgtagacaaccggatcgtggaattcatggagcacgaaaagcccaacaagctgaacgagggcaagctctttatctcgaccgaggagatgatggcgctcatcgtcggctacctggcgttcgtggttctcgggtctgccttcatgacggcctttgtgagtaagcctttcgagctcaagttcctgaagctcgtgcacaacatctttctcaccggtctgtccctgtacatggctagcgagtgcgcgcgccaggcctacctcggcggctacaagctctttggcaacccgatggagaagggtgccgagtctcacgccctgggcatggctagcattatctacgttttttacgtgagcaagttcctcgagtttcttgacacggtcttcatgatcctcggcaagaagtggaagcagctcagctttcttcacgtctaccaccacgcgagcatcagcttcatctggggcattatcgcccgttttgcgcccggtggcgacgcgtacttttccaccatcctcaacagcagcgtgcatgtcgtgctctacggctactacgcctcgaccacgctcggctacaccttcatgcgcccgctgcgcccgtacattactaccatccagctcacgcagttcatggccatggtcgtccagtccgtctatgactactacaacccttgcgactacccgcagcccctcgtcaagctactcttctggtacatgctcaccatgctcggccttttcggcaacttcttcgtgcagcagtacctcaagcccaaggcgcctaagaagcaaaagaccatc

(19) United States
(12) Patent Application Publication
(10) Pub. No.: US 2024/0182934 A1
(43) Pub. Date: Jun. 6, 2024

(54) OIL COMPOSITIONS WITH ENGINEERED LIPID PROFILES AND METHODS OF PRODUCING SAME

(71) Applicant: MARA Renewables Corporation, Dartmouth (CA)

(72) Inventors: Holly Rasmussen, Dartmouth (CA); David Woodhall, Herring Cove (CA); Jeremy Benjamin, Mineville (CA); Denise Muise, Cole Harbour (CA); Roberto E. Armenta, Dartmouth (CA)

(21) Appl. No.: 18/525,184

(22) Filed: Nov. 30, 2023

Related U.S. Application Data
(60) Provisional application No. 63/429,852, filed on Dec. 2, 2022.

(51) Int. Cl. C12P 7/6431; C12N 1/20; C12N 9/02; C12N 9/10; C12N 15/52; C12P 7/6432

(52) U.S. Cl. CPC C12P 7/6431; C12N 1/205; C12N 9/0071; C12N 9/1029; C12N 15/52; C12P 7/6432; C12Y 114/19001; C12Y 114/19006

(57) ABSTRACT

Provided herein are engineered microorganisms comprising one or more genes involved in fatty acid metabolism. For example, provided is an engineered microorganism comprising a first nucleic acid sequence encoding an elongase and a second nucleic acid sequence encoding a desaturase, wherein the first and second nucleic acid sequences are operably linked to promoter. Also provided are methods of making and using the engineered microorganisms. Also provided are microbial oils comprising fatty acids, wherein the fatty acids comprise C20:3(n-6) (di-homo-γ-linoleic acid) and C20:5(n-3) eicosapentaenoic acid (EPA). In addition, provided herein are methods of promoting conversion of saturated fatty acids to unsaturated fatty acids by transforming microorganisms with nucleic acid encoding polypeptides involved in the fatty acid synthesis pathway to generated increased conversion of saturated fatty acids to unsaturated fatty acids.

### CROSS REFERENCE TO RELATED APPLICATIONS

This application claims priority from U.S. Provisional Application No. 63/429,852, filed Dec. 2, 2022, the entire disclosure of which is incorporated herein by reference.

### SEQUENCE LISTING

This application contains a Sequence Listing which has been filed electronically in .xml format and is hereby incorporated by reference in its entirety. Said .xml copy, created on Nov. 29, 2023, is entitled “MAR-025US-WO-095523-1416607-1412622.xml”, and is 36 kilobytes in size. It is hereby stated that the information recorded on the computer readable form is identical to the written sequence listing and does not include matter which goes beyond the disclosure in the application as filed.

### BACKGROUND

Certain microorganisms produce oil as a result of two parallel fatty acid synthesis pathways: the classical fatty acid synthesis (FAS) pathway and the polyunsaturated fatty acid (PUFA) synthase pathway. Medium chain fatty acids like myristic (C14:0) and palmitic acid (C16:0) are generally produced from the FAS pathway and long chain polyunsaturated fatty acids (LC-PUFA) like docosahexaenoic acid (DHA, C22:6 n-3) and docosapentaenoic acid (DPA, C22:5 n-6) are generally produced from the PUFA synthase pathway. The resulting fatty acid profile, however, varies greatly across microorganisms, depending on the relative activity of these parallel pathways.

### BRIEF SUMMARY

Provided herein are engineered microorganisms comprising one or more heterologous nucleic acids encoding polypeptides involved in fatty acid metabolism. For example, provided is an engineered microorganism comprising a first nucleic acid sequence encoding an elongase and a second nucleic acid sequence encoding a desaturase, wherein the first and second nucleic acid sequences are operably linked to a promoter. Also provided are methods of making and using the engineered microorganisms. Also provided are microbial oils comprising fatty acids, wherein the fatty acids comprise C20:3(n-6) (di-homo-γ-linoleic acid) and C20:5(n-3) eicosapentaenoic acid (EPA). In addition, provided herein are methods of promoting conversion of saturated fatty acids to unsaturated fatty acids by transforming microorganisms with one or more nucleic acids that encode polypeptides involved in the fatty acid synthesis pathway to generated increased conversion of saturated fatty acids to unsaturated fatty acids.

### BRIEF DESCRIPTION OF THE DRAWINGS

FIG. 1 is a schematic showing a pathway for fatty acid modifications of C16:0 from the FAS pathway.

FIG. 2 is a series of schematics showing constructs transformed into WT T18 or derivative strains for production of different fatty acids. Δ9 desaturase, α-tubulin and PUFA synthase subunit B promoters, terminators (1 kb upstream and downstream of native genes, respectively), and internal coding regions are used as homology arms for homologous recombination.

FIG. 3A is a schematic of WT T18 and its homologous recombinant constructs (95 and 116) and shows a Southern blot strategy for confirmation of homologous recombinants at Δ9 desaturase for WT T18 and 95 series and 116 series transformants. FIG. 3B are images of Southern blots indicating all transformants demonstrate homologous recombination at the Δ9 site with one heterozygous transformant: 116-2. Probe is the region upstream of Δ9 desaturase promoter. Blots indicate all transformants are homologous recombinants at Δ9 desaturase site.

FIG. 4 is an image of a Southern blot of WT T18 and 12 transformants from the 95 series (shown in FIG. 2 and FIG. 3A). Genomic DNA (gDNA) for each transformant was digested with PstI. Blot was probed with a neomycin sequence to detect neomycin resistance gene in transformants. All transformants indicate the presence of neomycin resistance (neo-R) gene. The neo-R gene is absent in WT T18.

FIG. 5 is a graph showing fatty-acid methyl esters (FAMEs) analysis of a freeze-dried biomass for WT T18 and select 95 series transformants. See also FIG. 1 for fatty acid legend. Strains were grown in flask with 25 mL WDL medium to glucose depletion, approximately 7 days. Pellets were harvested and freeze-dried. Values are expressed as total fatty-acid present in mg/g dried biomass.

FIG. 6 is a graph showing the same FAME analysis of the 95 series transformants as FIG. 5 but expressed as a percent of the total-fatty acid profile. See also FIG. 1 for fatty acid legend.

FIG. 7 is a graph showing FAMEs analysis for freeze-dried biomass of strain 95-1 grown in regular WDL media or in WDL containing low nitrogen. Values expressed as total fatty-acid present in mg/g dry biomass. See also FIG. 1 for fatty acid legend.

FIG. 8 is a graph showing FAMEs analysis of samples taken during fermentation of transformant 95-1. See also FIG. 1 for fatty acid legend.

FIG. 9 is a Southern blot of luciferase transformants targeting the native Δ9 desaturase site.

FIG. 10 is a table showing select FAMEs data for luciferase transformants showing complete absence of C16:1 n-7, C18:1 n-9, and C18: 1 n-7 in transformant Δ12 luciferase 4.

FIG. 11 is a graph of FAMEs data showing fatty-acid content of 57 series transformants compared to WT T18 in mg/g dry biomass. See also FIG. 1 for fatty acid legend.

FIG. 12 is a table showing select FAMEs data for 57 series transformants showing increases in C16:1 n-7 and C18:1 n-7 over WT T18.

FIG. 13 is a graph of FAMEs data showing fatty-acid content of 84 series transformants compared to WT T18 in mg/g dry biomass. See also FIG. 1 for fatty acid legend.

FIG. 14 is a table showing select FAMEs data for 84 series transformants showing increases in C16:1 n-7 and C18:1 n-7 over WT T18.

FIG. 15 is a graph of fatty-acid methyl esters (FAMEs) analysis of freeze-dried biomass for WT T18 and 116 series transformants. See also FIG. 1 for fatty acid legend. Transformants were grown in a flask with 25 mL medium to glucose depletion, approximately 7 days. Pellets were harvested and freeze-dried. Values are presented as total fatty-acid present in mg/g dry biomass.

FIG. 16A provides schematics of WT T18 and a construct of its homologous recombinant with pHR47 at the α-tubulin locus. FIG. 16B shows a Southern blot comparing WT, 95-1 and recombinants in the 110 series. Probe is for region downstream of α-tubulin terminator. 110-1, 3, 4, 9, and 10 are probable α-tubulin double knockouts based on larger fragment size shown in the Southern blot.

FIG. 17 is a graph of fatty-acid methyl esters (FAMEs) analysis of freeze-dried biomass for WT T18, 95-1 parent and 110 series transformants. See also FIG. 1 for fatty acid legend. Transformants were grown in flask with 25 mL medium to glucose depletion, approximately 7 days. Pellets were harvested and freeze-dried. Values are presented as total fatty-acid present in mg/g dry biomass.

FIG. 18 is a graph of fatty-acid methyl esters (FAMEs) analysis of freeze-dried biomass for WT T18 and 67-1 from cultures fed with different free fatty-acid species. Values are total fatty-acid present in mg/g dry biomass. Conversion of ALA to EPA and GLA to ARA is seen in 67-1. See also FIG. 1 for fatty acid legend.

FIG. 19A provides schematics of WT T18 and a construct of its homologous recombinant with pHR47 at subB locus in the 113 series. FIG. 19B is a Southern blot comparing WT T18 and the homologous recombination of construct in pHR52 at subB locus in 113 series transformants. Probe is for region upstream of subB promoter. 113-4 is a double knockout at subB.

FIG. 20 is a graph of fatty-acid methyl esters (FAMEs) analysis of freeze-dried biomass for WT T18 and 113 series transformants. Values are presented as total fatty-acid present in mg/g dry biomass. See also FIG. 1 for fatty acid legend.

FIG. 21A provides a schematic for WT T18 and for a construct for a homologous recombinant in which pHR58 is at subB locus in the 121 series. FIG. 21B is a Southern blot showing WT-T18 and transformants with the homologous recombinations. Probe is for region downstream of subB promoter. 121-1 is a double knockout at subB locus.

FIG. 22 is a graph of fatty-acid methyl esters (FAMEs) analysis of freeze-dried biomass for 121-1 from cultures fed with different free fatty-acid (FFA) species (0.5 mM FFA in each culture). Values are presented as total fatty-acid present in mg/g dry biomass. Conversion of linoleic acid (LA) to α-linoleic acid (ALA) is seen in each culture. See also FIG. 1 for fatty acid legend.

FIG. 23 is a graph of the FAMEs results for 121-1 shown in FIG. 22 expressed as percent total fatty-acid (% TFA). See also FIG. 1 for fatty acid legend.

FIG. 24 is a graph showing increased substrate conversion by A12 desaturase (at either the α-tubulin site or the Δ9 desaturase site as shown above the graph) when expressed in same open-reading frame as C16 elongase and Δ9 desaturase rather than at a discrete site.

FIG. 25A is a schematic showing WT T18 and homologous recombinants in which subB is replaced with pHR64 or pHR62. FIG. 25B is a Southern blot comparing WT T18 with transformants in which subB replaced with pHR64 or pHR62. Probe is specific for region downstream of subB TR. 127-3 and 129-1 appear to have recombination events at sub and could be subB double knockouts but they lack the expected fragment size of a double knockout. 129-2 appears to be a double knockout at subB with the expected fragment size.

FIG. 26 is a graph of fatty-acid methyl esters (FAMEs) analysis of freeze-dried biomass for 116-5 parent and 127 series transformants. Values are presented as total fatty-acid present in mg/g dry biomass. See also FIG. 1 for fatty acid legend.

FIG. 27 is a graph of fatty-acid methyl esters (FAMEs) analysis of freeze-dried biomass for 116-5 parent and 129 series transformants. Values are presented as total fatty-acid present in mg/g dry biomass. See also FIG. 1 for fatty acid legend.

FIG. 28 is a graph of fatty-acid methyl esters (FAMEs) analysis of freeze-dried biomass for 121-1 passaged axenic strains 121-1-S and 121-1-F. Values are presented as total fatty-acid present in mg/g dry biomass. See also FIG. 1 for fatty acid legend.

FIG. 29 is a graph of fatty-acid methyl esters (FAMEs) analysis of freeze-dried biomass for 127-3 and 129-2 passaged axenic transformants 127-3-T, 127-3-W, 127-3-R, and 127-3-P (127-3-T, W, R+P), and 129-2-T, 129-2-W, 129-2-R, and 129-2-P (129-2-T, W, R+P). Values are presented as total fatty-acid present in mg/g dry biomass. See also FIG. 1 for fatty acid legend.

FIG. 30 is a graph of the same FAMEs results for 127-3 and 129-2 passaged axenic transformants as shown in FIG. 28 expressed as % TFA. See also FIG. 1 for fatty acid legend.

FIG. 31 is a graph of fatty-acid methyl esters (FAMEs) analysis of freeze-dried biomass for WT T18 and 164 series transformants. See also FIG. 1 for fatty acid legend. Transformants were grown in flask with 25 mL medium to glucose depletion, approximately 5 days. Pellets were harvested and freeze-dried. Values are presented as total fatty-acid present in mg/g dry biomass.

FIG. 32 is a graph of fatty-acid methyl esters (FAMEs) analysis of freeze-dried biomass for 167 series transformants. See also FIG. 1 for fatty acid legend. Transformants were grown in flask with 25 mL medium to glucose depletion, approximately 5 days. Pellets were harvested and freeze-dried. Values are presented as total fatty-acid present in mg/g dry biomass.

FIG. 33 is a graph of fatty-acid methyl esters (FAMEs) analysis of freeze-dried biomass for 165 series transformants. See also FIG. 1 for fatty acid legend. Transformants were grown in flask with 25 mL medium to glucose depletion, approximately 9 days. Pellets were harvested and freeze-dried. Values are presented as total fatty-acid present in mg/g dry biomass.

FIG. 34 is a graph of the results shown in FIG. 33 and expressed as % TFA. See also FIG. 1 for fatty acid legend.

FIG. 35 is a graph of fatty-acid methyl esters (FAMEs) analysis of freeze-dried biomass for 173 series transformants. See also FIG. 1 for fatty acid legend. Transformants 173-1 and 173-2 (173-1+2) were grown in flask in 25 mL 10% N WDL until glucose consumption stalled at ˜13 g/L glucose after 7 days. Transformant 173-3 was grown in flask with 25 mL 10% N WDL to glucose depletion, after 4 days. Pellets were harvested and freeze-dried. Values are presented as total fatty-acid present in mg/g dry biomass.

FIG. 36 is a graph of fatty-acid methyl esters (FAMEs) analysis of freeze-dried biomass for 173-1 and 173-2 passaged axenic transformants 173-1-T, 173-1-W, 173-1-R, and 173-1-P (173-1-T, W, R+P), and 173-2-W, 173-2-R, and 173-2-P (173-2-W, R+P). Values are presented as total fatty-acid present in mg/g dry biomass. See also FIG. 1 for fatty acid legend.

FIG. 37 is a graph of the 173-1 and 173-2 passaged axenic transformants shown in FIG. 33 and expressed as % TFA. See also FIG. 1 for fatty acid legend.

FIG. 38 is a graph of RT-qPCR results for differential expression of five elongases in WT T18 versus 173-1-R and 173-2-R.

FIG. 39 is a graph of fatty-acid methyl esters (FAMEs) analysis of freeze-dried biomass for 173-1-R ALE 2-3 C1-8. Values are presented as total fatty-acid present in mg/g dry biomass. See also FIG. 1 for fatty acid legend.

FIG. 40 is a graph of the FAMEs results for 173-1-R ALE 2-3 C1-8 that are shown in FIG. 39 and expressed as % TFA. See also FIG. 1 for fatty acid legend.

FIG. 41 is a graph of fatty-acid methyl esters (FAMEs) analysis of freeze-dried biomass for 173-1-R and 173-1-R MUT1. Values are presented as total fatty-acid present in mg/g dry biomass. See also FIG. 1 for fatty acid legend.

FIG. 42 is a graph of FAMEs results for 173-1-R and 173-1-R MUT1 shown in FIG. 41 and expressed as % TFA. See also FIG. 1 for fatty acid legend.

FIG. 43 is a graph of fatty-acid methyl esters (FAMEs) analysis of freeze-dried biomass for 173-1-R and 173-1-R MUT5. Values are presented as total fatty-acid present in mg/g dry biomass. See also FIG. 1 for fatty acid legend.

FIG. 44 is a graph of FAMEs results for 173-1-R and 173-1-R MUT5 shown in FIG. 43 and expressed as % TFA. See also FIG. 1 for fatty acid legend.

FIG. 45 is a graph comparing 116 series, which has C16 elongase (Obl) 3′ to A9 PR with 136 series and 137 series transformations, which have A6 desaturase (Bty) and (23 desaturase (Obl) 3′ to Δ9 PR respectively. There is about a 10-fold reduction in the production of oleic acid in 136 and 137 series transformants compared to 116 series transformants. The reduced activity of C16 elongase (Obl) on C16:0 in 136 and 137 series results in accumulation of C16:0 to over 2.5 times amount in 116 series. See also FIG. 1 for fatty acid legend.

FIG. 46 is a graph showing FAMEs analysis of strains 180-1 and 180-2 in mg/g.

FIG. 47 is a graph showing FAMEs analysis of strains 183-3, 183-5, 183-6, 183-7, and 183-8 in mg/g.

FIG. 48 is a graph showing FAMEs analysis of strains 183-8-T, 183-8-W, 183-8-R, and 183-8-P in mg/g.

FIG. 49 is a graph showing FAMEs analysis of strains 190-1, 190-2, 190-3, 190-4, 190-5, 190-6 in mg/g.

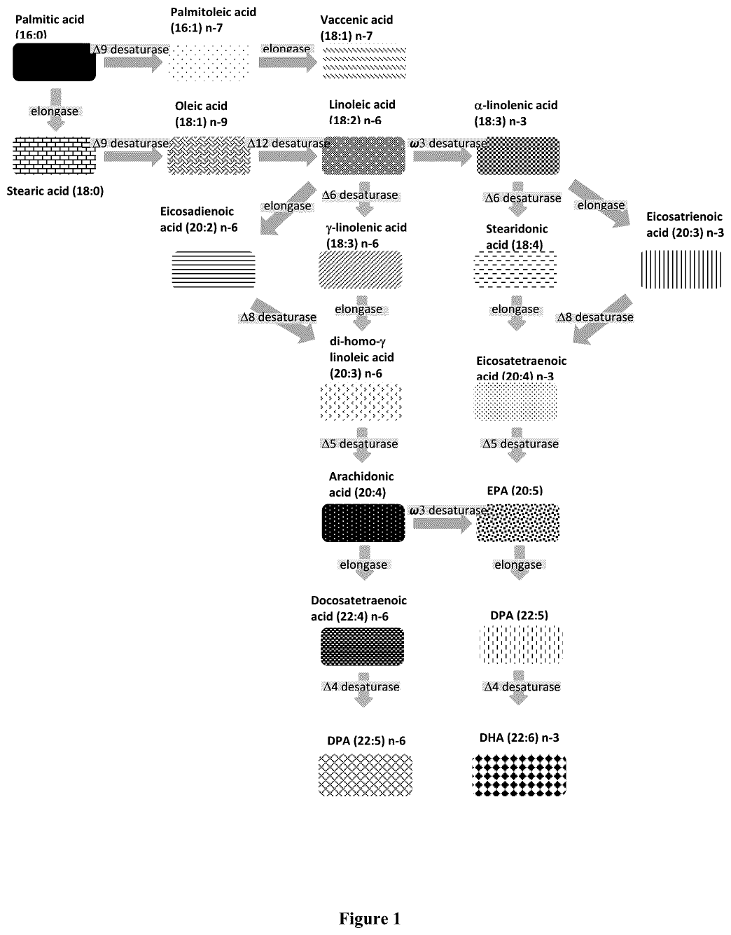

### DETAILED DESCRIPTION

Certain microorganisms, including Thraustochytrids, produce oil containing a variety of lipids, including fatty acids in various forms and amounts. As used herein, the term lipid includes phospholipids, free fatty acids, esters of fatty acids, triacylglycerols, sterols and sterol esters, carotenoids, xanthophylls (e.g., oxycarotenoids), hydrocarbons, and other lipids. Fatty acids are hydrocarbon chains that terminate in a carboxyl group, being termed unsaturated if they contain at least one carbon-carbon double bond and polyunsaturated when they contain multiple carbon-carbon double bonds. For example, microorganisms can produce (i) short-chain fatty acids (SCFA), which are fatty acids with aliphatic tails of fewer than six carbons (e.g., butyric acid); (ii) medium-chain fatty acids (MCFA), which are fatty acids with aliphatic tails of 6-12 carbons; (iii) long-chain fatty acids (LCFA), which are fatty acids with aliphatic tails of greater than 13 carbons. Various microorganisms produce varying types and amounts of these fatty acids. Provided herein are microorganisms and methods that shift production of these fatty acids away from medium-chain fatty acids produced by the FAS pathway to long-chain fatty acids produced by the PUFA synthase pathway. Fatty acid synthesis (FAS) is defined as the creation of fatty acids from acetyl-CoA and NADPH through the action of enzymes called fatty acid synthases. The PUFA synthase pathway enables the synthesis of polyunsaturated fatty acids de novo from malonyl-CoA by large multi-domain, multi-subunit enzymes. The major end-product of the FAS pathway is palmitate, while the major end-product of the PUFA synthases are PUFAs such as DHA and DPA.

Microorganisms can be used for commercial production of lipids for applications including nutritional supplements, animal feed, or biofuels. Increased mono- or poly-unsaturated fatty acids (MUFAs and PUFAs) at the expense of saturated fatty acids result in increased oil flowability making for easier downstream handling. Shorter carbon chains or MUFAs may be desired for biofuel applications. High Ω-3 or Ω-6 content may be desired for nutritional applications. In addition to targeting specific oil profiles, producing the same profiles with the same methods consistently is imperative in an industrial setting. Provided herein are genetically modified strains capable of producing oil profiles consistent in fermentation.

Eukaryotic microorganisms useful for producing the provided microbial oils and biomasses include, but are not limited to, microorganisms selected from the genus Oblongichytrium (Obl), Aurantiochytrium, Thraustochytrium, Schizochytrium, and Ulkenia or any mixture thereof. Optionally, the eukaryotic microorganism is the same as the microorganism deposited with the American Type Culture Collection (ATCC), 10801 University Boulevard, Manassass VA, 20110-2209, on Oct. 6, 2004, having ATCC assigned Accession No. PTA-6245. This deposit is exemplary and was made merely as a convenience for those of skill in the art and is not an admission that a deposit is required for patentability. As used throughout, the terms T18, and WT T18 are used interchangeably and refer to the same microorganism, ATCC Accession No. PTA-6245.

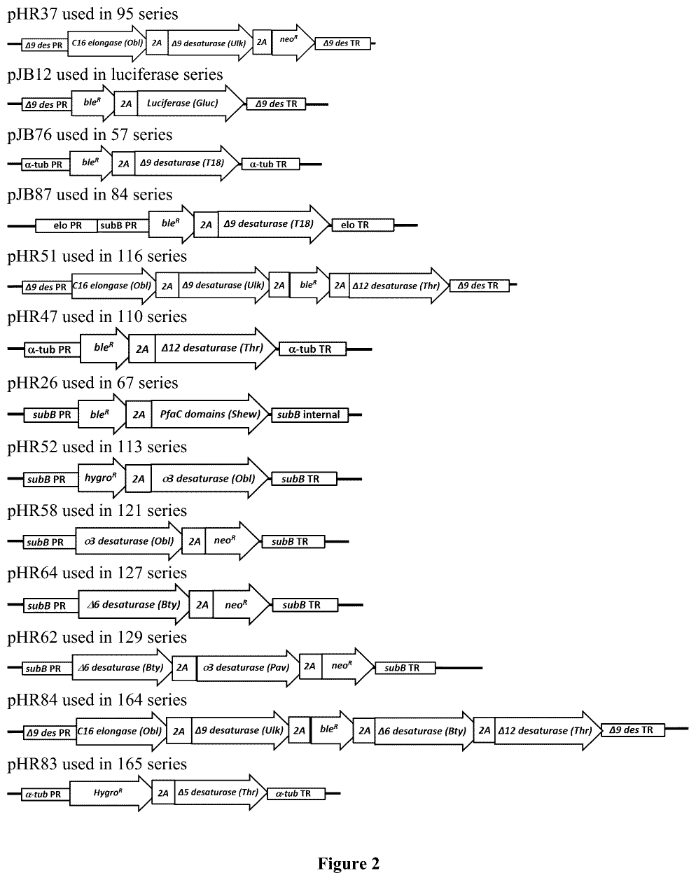

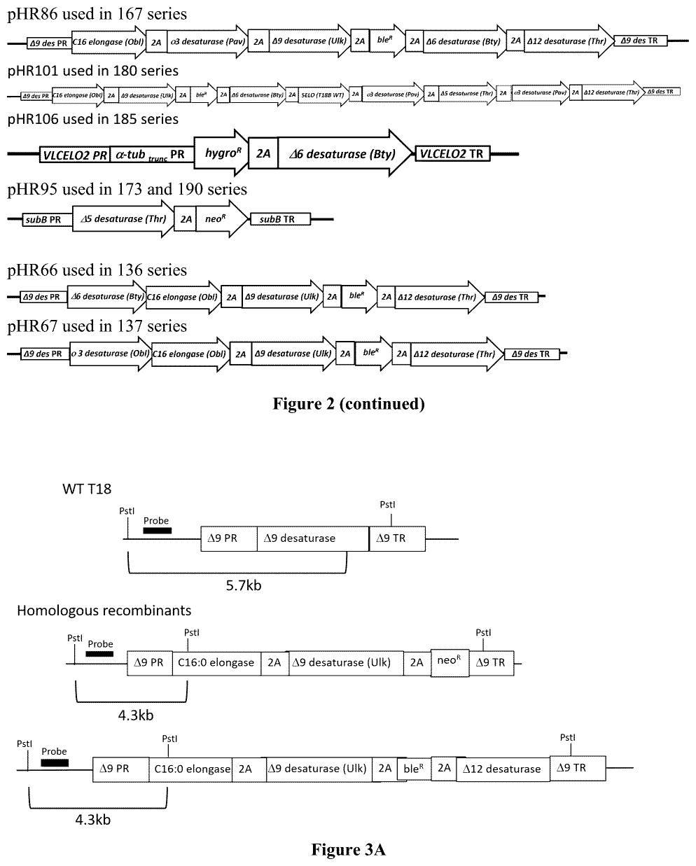

Provided herein are engineered microorganisms comprising one or more heterologous nucleic acids encoding polypeptides involved in fatty acid metabolism. By heterologous nucleic acid is meant a nucleic acid sequence not normally found in the given cell in nature. The heterologous nucleic acid can be foreign to its host cell, naturally found but present at an unnatural quantity in the cell (e.g., greater of lesser quantity than naturally found in the cell), or naturally found in the host cell but positioned outside of its natural locus. By way of example, provided is an engineered microorganism comprising a first heterologous nucleic acid sequence encoding an elongase and a second heterologous nucleic acid sequence encoding a desaturase, wherein the first and second nucleic acid sequences are operably linked to promoter. The promoter can be a Δ9 desaturase, Δ5 desaturase, subB or α-tubulin promoter. The promoter can be used in its native genomic location or outside of its original, native genomic location. Thus, the promoter, e.g., the Δ9 desaturase promoter, can be located in its native position in the genome of the microorganism. Optionally, the promoter, e.g., the Δ9 desaturase promoter, and the nucleic acids encoding the fatty acid metabolism polypeptides are located on a heterologous construct. (See, e.g., FIG. 2). The desaturase can be a Δ9 desaturase, which can be a Thraustochytrium sp. or Ulkenia sp. Δ9 desaturase. The elongase can be, for example, a Delta 5 elongase or a C16:0 elongase, which can be a Oblongichytrium sp. elongase. Optionally, the first and second nucleic acids disrupt the microorganism's endogenous Δ9 desaturase sequence.

Optionally, the provided microorganisms comprise several nucleic acids encoding polypeptides involved in fatty acid metabolism. Thus, the provided microorganisms can further include an (23 desaturase, which can be a Oblongichytrium sp. 523 desaturase. The engineered microorganisms can include a heterologous nucleic acid encoding a Δ12 desaturase, which can be a Thraustochytrium sp. desaturase. The engineered microorganisms can include a Δ6 desaturase, which can be a Botryochytrium sp. Δ6 desaturase. The engineered microorganisms can also include a Δ5 desaturase, which can be a Thraustochytrium sp. Δ5 desaturase.

The engineered microorganisms can be engineered to include a number of nucleic acids using constructs including additional sequences such as promoters, selectable markers or resistance genes, terminators, linking sequences, and the like. In some instances, the constructs include a resistance gene against zeomycin, bleomycin, neomycin, hygromycin, or G418. Optionally, the engineered microorganisms comprise a zeocin resistance gene. Optionally, the engineered microorganisms comprise one or more 2A sequences. The engineered microorganisms can include a reporter gene. Optionally, the reporter gene is luciferase. As discussed, the engineered microorganisms can comprise the nucleic acids with one or more tubulin promoters, one or more tubulin terminators, or both one or more tubulin promoters and one or more tubulin terminators. Optionally, the nucleic acid comprises one or more PUFA synthase subunit B promoters, one or more PUFA synthase subunit B terminators, or both one or more PUFA synthase subunit B promoters and one or more PUFA synthase subunit B terminators.

The engineered microorganisms can be modified to include a nucleic acid sequence or construct comprising different nucleic acids, e.g., one or more promoters, nucleic acids encoding polypeptides involved in fatty acid synthesis, terminators, linking sequences, and the like. By way of example, an engineered microorganism can include any combination or sequence of nucleic acids as described herein. Examples of constructs are shown in FIG. 2. Thus, an engineered microorganism can include a Δ9 desaturase promoter, a nucleic acid sequence encoding a C16 elongase, a 2A sequence, a nucleic acid encoding a Δ9 desaturase, a 2A sequence, a neomycin resistance gene, and a Δ9 desaturase terminator. An engineered microorganism can include a Δ9 desaturase promoter, a bleomycin resistance gene, a 2A sequence, a luciferase gene, and a Δ9 desaturase terminator. An engineered microorganism can include an α-tubulin promoter, a bleomycin resistance gene, a 2A sequence, a nucleic acid encoding a Δ9 desaturase and an α-tubulin terminator. An engineered microorganism can include an elongase promoter, a subB promoter, a bleomycin resistance gene, a 2A sequence, a nucleic acid encoding a Δ 9 desaturase and an elongase terminator. An engineered microorganism can include a Δ9 desaturase promoter, a nucleic acid sequence encoding a C16 elongase, a 2A sequence, a nucleic acid encoding a Δ9 desaturase, a 2A sequence, a bleomycin resistance gene, a 2A sequence, a nucleic acid encoding a Δ12 desaturase, and a Δ9 desaturase terminator. An engineered microorganism can include an alpha-tubulin promoter, a bleomycin resistance gene, a 2A sequence, a Δ12 desaturase, and an alpha-tubulin terminator. An engineered microorganism can include a subB promoter, a bleomycin resistance gene, a 2A sequence, a nucleic acid encoding PfaC domain, and a subB internal sequence. An engineered microorganism can include a subB promoter, a hygromycin resistance gene, a 2A sequence, a nucleic acid encoding an Ω-3 desaturase, and a subB terminator. An engineered microorganism can include a subB promoter, a nucleic acid encoding an Ω-3 desaturase, a 2A sequence, a neomycin resistance gene and a subB terminator. An engineered microorganism can include an α-tubulin promoter, a hygromycin resistance gene, a 2A sequence, a nucleic acid encoding a Δ5 desaturase, and an α-tubulin terminator. An engineered microorganism can include an α-tubulin promoter, a nucleic acid encoding a Δ5 desaturase, a 2A sequence, a neomycin resistance gene, and an α-tubulin terminator. An engineered microorganism can include a subB promoter, a nucleic acid encoding a Δ6 desaturase, a 2A sequence, a neomycin resistance gene and a subB terminator. An engineered microorganism can include a subB promoter, a nucleic acid encoding a Δ6 desaturase, a 2A sequence, a nucleic acid encoding an Ω-3 desaturase, a 2A sequence, a neomycin resistance gene and a subB terminator. An engineered microorganism can include a Δ9 desaturase promoter, a nucleic acid sequence encoding a C16 elongase, a 2A sequence, a nucleic acid encoding a Δ9 desaturase, a 2A sequence, a bleomycin resistance gene, a 2A sequence, a nucleic acid encoding a Δ6 desaturase, a 2A sequence, a nucleic acid encoding a Δ12 desaturase, and a Δ9 desaturase terminator. An engineered microorganism can include a Δ9 desaturase promoter, a nucleic acid sequence encoding a C16 elongase, a 2A sequence, a nucleic acid encoding an Ω-3 desaturase, a 2A sequence, a nucleic acid encoding a Δ9 desaturase, a 2A sequence, a bleomycin resistance gene, a 2A sequence, a nucleic acid encoding a Δ6 desaturase, a 2A sequence, a nucleic acid encoding a Δ12 desaturase, and a Δ9 desaturase terminator. An engineered microorganism can include a Δ9 desaturase promoter, a nucleic acid sequence encoding a Δ6 desaturase, a nucleic acid encoding a C16 elongase, a 2A sequence, a nucleic acid encoding a Δ9 desaturase, a 2A sequence, a bleomycin resistance gene, a 2A sequence, a nucleic acid encoding a Δ12 desaturase, and a Δ9 desaturase terminator. An engineered microorganism can include a Δ9 desaturase promoter, a nucleic acid sequence encoding an Ω-3 desaturase, a nucleic acid encoding a C16 elongase, a 2A sequence, a nucleic acid encoding a Δ9 desaturase, a 2A sequence, a bleomycin resistance gene, a 2A sequence, a nucleic acid encoding a Δ12 desaturase, and a Δ9 desaturase terminator. An engineered microorganisms can include a Δ9 desaturase promoter, a nucleic acid encoding a C16 elongase, a 2A sequence, a nucleic acid encoding a Δ9 desaturase, a 2A sequence, a bleomycin resistance gene, a 2A sequence, a nucleic acid sequence encoding a Δ6 desaturase, a 2A sequence, a nucleic acid sequence encoding a 45 elongase, a nucleic acid sequence encoding an Ω-3 desaturase, a 2A sequence, a Δ5 desaturase, a 2A sequence, a nucleic acid sequence encoding an Ω-3 desaturase, a 2A sequence, a nucleic acid sequence encoding a Δ12 desaturase, and a Δ9 desaturase terminator.

The term transformation, as used herein refers to a process by which a heterologous nucleic acid molecule (e.g., a vector or recombinant nucleic acid molecule) is introduced into a recipient cell or microorganism. The heterologous nucleic acid molecule may or may not be integrated into (i.e., covalently linked to) chromosomal DNA making up the genome of the host cell or microorganism. For example, the heterologous polynucleotide may be maintained on an episomal element, such as a plasmid. Alternatively or additionally, the heterologous polynucleotide may become integrated into a chromosome so that it is inherited by daughter cells through chromosomal replication. Methods for transformation include, but are not limited to, calcium phosphate precipitation; Ca²⁺ treatment; fusion of recipient cells with bacterial protoplasts containing the recombinant nucleic acid; treatment of the recipient cells with liposomes containing the recombinant nucleic acid; DEAE dextran; fusion using polyethylene glycol (PEG); electroporation; magnetoporation; biolistic delivery; retroviral infection; lipofection; and micro-injection of DNA directly into cells.

The term transformed, as used in reference to cells, refers to cells that have undergone transformation as described herein such that the cells carry heterologous genetic material (e.g., a recombinant nucleic acid). The term transformed can also or alternatively be used to refer to microorganisms, strains of microorganisms, tissues, organisms, and the like that contain heterologous genetic material.

The term introduce as used herein with reference to introduction of a nucleic acid into a cell or organism, is intended to have its broadest meaning and to encompass introduction, for example by transformation methods (e.g., calcium-chloride-mediated transformation, electroporation, particle bombardment), and also introduction by other methods including transduction, conjugation, and mating. Optionally, a construct is utilized to introduce a nucleic acid into a cell or organism. As used herein, the term transformant, refers to a cell, microorganism, strain of microorganism, tissue, organism, and the like that contains nucleic acids that have been introduced or transformed into the cell, microorganism, strain of microorganism, tissue, organism, and the like.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

As used herein, the terms promoter, promoter element, and regulatory sequence refer to a polynucleotide that regulates expression of a selected polynucleotide sequence operably linked to the promoter and that effects expression of the selected polynucleotide sequence in cells. The term Thraustochytrium promoter, as used herein, refers to a promoter that naturally occurs in a Thraustochytrium cell. In some embodiments, a promoter element is or comprises untranslated regions (UTR) in a position 5′ of coding sequences. 5′ UTRs form part of the mRNA transcript and so are an integral part of protein expression in eukaryotic organisms. Following transcription 5′UTRs can regulate protein expression at both the transcription and translation levels.

As used herein, the term terminator refers to a polynucleotide that abrogates expression of, targets for maturation of (e.g., adding a polyA tail), or imparts mRNA stability to a selected polynucleotide sequence operably linked to the terminator in cells. A terminator sequence may be downstream of a stop codon in a nucleic acid. The term Thraustochytrium terminator, as used herein, refers to a terminator that naturally occurs in a Thraustochytrium cell. Provided herein are also nucleic acid constructs that include nucleic acid sequences encoding xylose isomerase, xylulose kinase and xylose transporter as well as promoters, terminators, selectable markers, 2A peptides or any combination thereof.

The phrase selectable marker, as used herein, refers either to a nucleotide sequence, e.g., a gene, that encodes a product (polypeptide) that allows for selection, or to the nucleotide sequence product (e.g., polypeptide) itself. The term selectable marker is used herein as it is generally understood in the art and refers to a marker whose presence within a cell or organism confers a significant growth or survival advantage or disadvantage on the cell or organism under certain defined culture conditions (selective conditions). For example, the conditions may be the presence or absence of a particular compound or a particular environmental condition such as increased temperature, increased radiation, presence of a compound that is toxic in the absence of the marker, etc. The presence or absence of such compound(s) or environmental condition(s) is referred to as a selective condition(s). By growth advantage is meant either enhanced viability (e.g., cells or organisms with the growth advantage have an increased life span, on average, relative to otherwise identical cells lacking the trait or condition that conveys the growth advantage), increased rate of proliferation (also referred to herein as growth rate) relative to otherwise identical cells or organisms, or both. In general, a population of cells having a growth advantage will exhibit fewer dead or nonviable cells and/or a greater rate of cell proliferation than a population of otherwise identical cells lacking the growth advantage. Although typically a selectable marker will confer a growth advantage on a cell, certain selectable markers confer a growth disadvantage on a cell, e.g., they make the cell more susceptible to the deleterious effects of certain compounds or environmental conditions than otherwise identical cells not expressing the marker. Antibiotic resistance markers are a non-limiting example of a class of selectable markers that can be used to select cells that express the marker. In the presence of an appropriate concentration of antibiotic (selective condition), such a marker confers a growth advantage on a cell that expresses the marker. Thus, cells that express the antibiotic resistance marker are able to survive and/or proliferate in the presence of the antibiotic while cells that do not express the antibiotic resistance marker are not able to survive and/or are unable to proliferate in the presence of the antibiotic.

Examples of selectable markers include common bacterial antibiotics, such as but not limited to ampicillin, kanamycin and chloramphenicol, as well as selective compounds known to function in microalgae; examples include rrnS and AadA (Aminoglycoside 3′-adenylytranferase), which may be isolated from E. coli plasmid R538-1, conferring resistance to spectinomycin and streptomycin, respectively in E. coli and some microalgae (Hollingshead and Vapnek, Plasmid 13:17-30, 1985; Meslet-Cladière and Vallon, Eukaryot Cell. 10(12):1670-8 2011). Another example is the 23S RNA protein, rrnL, which confers resistance to erythromycin (Newman, Boynton et al., Genetics, 126:875-888 1990; Roffey, Golbeck et al., Proc. Natl Acad. Sci. USA, 88:9122-9126 1991). Another example is Ble, a GC rich gene isolated from Streptoalloteichus hindustanus that confers resistance to zeocin (Stevens, Purton et al., Mol. Gen. Genet., 251:23-30 1996). Aph7 is yet another example, which is a Streptomyces hygroscopicus-derived aminoglycoside phosphotransferase gene that confers resistance to hygromycin B (Berthold, Schmitt et al., Protist 153(4):401-412 2002). Additional examples include AphVIII, a Streptomyces rimosus derived aminoglycoside 3′-phosphotransferase type VIII that confers resistance to Paromomycin in E. coli and some microalgae (Sizova, Lapina et al., Gene 181(1-2):13-18 1996; Sizova, Fuhrmann et al., Gene 277(1-2):221-229 2001); Nat & Sat-1, which encode nourseothricin acetyl transferase from Streptomyces noursei and streptothricin acetyl transferase from E. coli, which confer resistance to nourseothricin (Zaslavskaia, Lippmeier et al., Journal of Phycology 36(2):379-386, 2000); Neo, an aminoglycoside 3′-phosphotransferase, conferring resistance to the aminoglycosides; kanamycin, neomycin, and the analog G418 (Hasnain, Manavathu et al., Molecular and Cellular Biology 5(12):3647-3650, 1985); and Cry1, a ribosomal protein S14 that confers resistance to emetine (Nelson, Savereide et al., Molecular and Cellular Biology 14(6):4011-4019, 1994).

Other selectable markers include nutritional markers, also referred to as auto- or auxo-trophic markers. These include photoautotrophy markers that impose selection based on the restoration of photosynthetic activity within a photosynthetic organism. Photoautotrophic markers include, but are not limited to, AtpB, TscA, PetB, NifH, psaA and psaB (Boynton, Gillham et al., Science 240(4858): 1534-1538 1988; Goldschmidt-Clermont, Nucleic Acids Research 19(15): 4083-4089, 1991; Kindle, Richards et al., PNAS, 88(5): 1721-1725, 1991; Redding, MacMillan et al., EMBO J 17(1):50-60, 1998; Cheng, Day et al., Biochemical and Biophysical Research Communications 329(3):966-975, 2005). Alternative or additional nutritional markers include ARG7, which encodes argininosuccinate lyase, a critical step in arginine biosynthesis (Debuchy, Purton et al., EMBO J 8(10):2803-2809, 1989); NITI, which encodes a nitrate reductase essential to nitrogen metabolism (Fernández, Schnell et al., PNAS, 86(17):6449-6453, 1989); THI10, which is essential to thiamine biosynthesis (Ferris, Genetics 141(2):543-549, 1995); and NIC1, which catalyzes an essential step in nicotinamide biosynthesis (Ferris, Genetics 141(2):543-549, 1995). Such markers are generally enzymes that function in a biosynthetic pathway to produce a compound that is needed for cell growth or survival. In general, under nonselective conditions, the required compound is present in the environment or is produced by an alternative pathway in the cell. Under selective conditions, functioning of the biosynthetic pathway, in which the marker is involved, is needed to produce the compound.

The phrase selection agent, as used herein refers to an agent that introduces a selective pressure on a cell or population of cells either in favor of or against the cell or population of cells that bear a selectable marker. For example, the selection agent is an antibiotic and the selectable marker is an antibiotic resistance gene. Optionally, zeocin is used as the selection agent.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated as appropriate. The sequence comparison algorithm then calculates the percent sequence identities for the test sequences relative to the reference sequence based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, WI); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al.). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

Also provided herein are methods of promoting conversion of saturated fatty acids to unsaturated fatty acids. The method includes transforming an oil-producing microorganism with a construct comprising a first nucleic acid encoding an elongase and a second nucleic acid encoding a desaturase, wherein the construct is inserted into the genome of the oil-producing microorganism at a location wherein expression of the encoded elongase and desaturase is controlled by a promoter native to the oil-producing microorganism; and culturing the transformed microorganisms under conditions to produce fatty acids, wherein the transformed microorganisms convert saturated fatty acids to unsaturated fatty acids greater than a control untransformed microorganism. Optionally, the native promoter is a Δ9 desaturase promoter. Optionally, the desaturase is a Δ9 desaturase. The Δ9 desaturase can be a Thraustochytrium sp. or Ulkenia sp. Δ9 desaturase. The elongase of these methods can be a Delta 5 elongase or a C16:0 elongase, which can be an Oblongichytrium sp. C16:0 elongase. Optionally, the construct further comprises a third nucleic acid encoding a Δ12 desaturase, which can be a Thraustochytrium sp. Δ12 desaturase. Optionally, the construct further comprises a fourth nucleic acid encoding a Δ6 desaturase, which can be a Botryochytrium sp. Δ6 desaturase.

The construct in these methods can further include additional nucleic acids, e.g., a nucleic acid encoding an Ω-3 desaturase, a nucleic acid encoding a Δ5 desaturase or both. Optionally, the Ω-3 desaturase is a Oblongichytrium sp. Ω-3 desaturase. Optionally, the Δ5 desaturase is a Thraustochytrium sp. Δ5 desaturase.

In the provided methods, the first and second nucleic acids can replace the microorganism's endogenous Δ9 desaturase-encoding sequence.

As described throughout, the constructs can include a number of nucleic acids encoding polypeptides included in fatty acid synthesis using constructs including additional sequences, such as promoters, selectable markers or resistance genes, terminators, linking sequences, and the like. For example, the construct can include a zeocin resistance gene; one or more 2A sequences; a reporter gene, e.g., luciferase; one or more tubulin promoters, one or more tubulin terminators, or both one or more tubulin promoters and one or more tubulin terminators; one or more PUFA synthase subunit B promoters, one or more PUFA synthase subunit B terminators, or both one or more PUFA synthase subunit B promoters and one or more PUFA synthase subunit B terminators; and combinations thereof.

The method can result in saturated fatty acids that are converted to unsaturated fatty acids are C16:0 and C18:0. The unsaturated fatty acids can be C18:1 (oleic acid), C18:2 (n-6) (linoleic acid), C18:3(n-3) (α-linoleic acid), C18:3(n-6) (γ-linoleic acid), or any combination thereof. The method can also result in transformed microorganisms that produce increased amounts of C20:3(n-6) (di-homo-γ-linoleic acid), C20:4(n-3) (eicosatetraenoic acid), C20:5(n-3) (EPA), and C22:5(n-3) (DPA-3) as compared to the control untransformed microorganism.

Also provided are microbial oils produced by the engineered microorganism described herein. The microbial oil can be produced by a microorganism selected from the group consisting of the genus Schizochytrium, Oblongichytrium, Aurantiochytrium and Thraustochytrium. Thus, provided herein are microbial oils and methods for making and using microbial oils. The oils include fatty acids in the form of monoglycerides, diglycerides, and triglycerides, as well as free fatty acids and phospholipids. Optionally, the microbial oil comprises at least 90% (e.g., 91%, 92%, 93%, 94%, 95%, 96%, 97%, 98%, or 99%) triglycerides. Optionally, the microbial oil comprises at least 95% triglycerides.

The oils also contain at least 85% (e.g., 86%, 87%, 88%, 89%, 90%, 91%, 92%, 93%, 94%, 95%, 96%, 97%, 98%, or 99%) by weight total fatty acids (TFA). Optionally, the oil contains between 85% to 99% total fatty acids by weight. Optionally, the microbial oil comprises 85% to 95% total fatty acids by weight. Optionally, the microbial oil comprises at least 90% total fatty acids by weight.

The percentages in reference to oils or total fatty acids are recited throughout by weight percent. For example, when a microbial oil comprises at least 90% total fatty acids, the oil contains at least 90% total fatty acids by weight of the oil. Also, total fatty acids contain specific fatty acids and percentages of the specific fatty acid are expressed throughout as weight % of the total fatty acids. For example, when the total fatty acids in the oil contain DHA, the amount of DHA is expressed as weight % of the total fatty acids. For example, the total fatty acids comprise at least 50% DHA by weight.

As described, the total fatty acids of the provided oils contain DHA. Optionally, the total fatty acids comprise at least 35%, at least 40%, or at least 45% DHA. Optionally, the total fatty acids comprise at least 50% DHA. Optionally, the total fatty acids include at least 60% DHA. Optionally, the total fatty acids include 50% to 70% DHA. Optionally, the total fatty acids comprise 60% to 70% DHA.

Provide herein are oil comprising C20:3(n-6) (di-homo-γ-linoleic acid) and C20:5(n-3) eicosapentaenoic acid (EPA). Thus, provided is a microbial oil comprising fatty acids, wherein the fatty acids comprise C20:3(n-6) (di-homo-γ-linoleic acid) and C20:5(n-3) eicosapentaenoic acid (EPA). The fatty acids can include 0.01% to 16% C20:3(n-6) (di-homo-γ-linoleic acid) (DLGA) or any percentage or range within 0.01% to 16% DLGA. The fatty acids can include 1-17% EPA or 5-17% EPA or any percentage or range within 1 to 17%.

The fatty acids in the oil can also include C14:0 (myristic acid) or C16:0 (hexadecanoic acid). Optionally, the fatty acids comprise 5-10% C14:0 (myristic acid) or any percentage or range within 5-10%. Optionally, the fatty acids comprise 13-22% of C16:0 (hexadecanoic acid) or any percentage or range within 13-22%.

The fatty acids in the oil can include C18 unsaturated fatty acids. Optionally, the fatty acids comprise 10-60% C18 unsaturated fatty acids. The fatty acids can include 10-45% C18:1 oleic acid. Optionally, the C18 unsaturated fatty acids include C18:2 (n-6) linoleic acid. For example, the fatty acids can include 0.01% to 40% linoleic acid or any percentage or range within 0.01% to 40%.

Optionally, the fatty acids in the oil further comprise C18:3(n-3) (α-linoleic acid), C18:3(n-6) (γ-linoleic acid), C20:4(n-3) (eicosatetraenoic acid), C20:5(n-3) (EPA), and C22:5(n-3) (docosapentaenoic acid (n-3) (DPA-3).

Optionally, the total fatty acids in the oil comprise less than 45%, 40%, 35%, 30%, or 25% saturated fatty acids (SFAs). Saturated fatty acids in the oils produced by the herein described method include, but are not limited to, C12:0 (lauric acid), C14:0 (myristic acid), C15:0 (pentadecanoic acid), C16:0 (palmitic acid), C17:0 (heptadecanoic acid), and C18:0 (stearic acid). Optionally, the total fatty acids comprise between 0.001% to 45% saturated fatty acids. Optionally, the total fatty acids in the oil comprises 10% and 45% saturated fatty acids (e.g., 10% and 40%, 10% and 30, 10% and 20%, 15% and 30%, 15% and 20%, 20% and 30%, or 20% and 25% saturated fatty acids). Thus, the microbial oil can include less than 35% saturated fatty acids. The microbial oil can include less than 30% saturated fatty acids. Optionally, the microbial oil comprises 0.001% to 35% saturated fatty acids.

Also provided are method for producing polyunsaturated fatty acids comprising providing the engineered microorganisms as described herein and culturing the engineered microorganism under conditions sufficient to produce the polyunsaturated fatty acids.

Culture medium as used in the described methods supplies various nutritional components, including a carbon source and a nitrogen source, for the microorganisms. Medium for culture can include any of a variety of carbon sources. Examples of carbon sources include fatty acids, lipids, glycerols, triglycerols, carbohydrates, polyols, amino sugars, and any kind of biomass or waste stream. Fatty acids include, for example, oleic acid. Carbohydrates include, but are not limited to, glucose, cellulose, hemicellulose, fructose, dextrose, xylose, lactulose, galactose, maltotriose, maltose, lactose, glycogen, gelatin, starch (corn or wheat), acetate, m-inositol (e.g., derived from corn steep liquor), galacturonic acid (e.g., derived from pectin), L-fucose (e.g., derived from galactose), gentiobiose, glucosamine, alpha-D-glucose-1-phosphate (e.g., derived from glucose), cellobiose, dextrin, alpha-cyclodextrin (e.g., derived from starch), and sucrose (e.g., from molasses). Polyols include, but are not limited to, maltitol, erythritol, and adonitol. Amino sugars include, but are not limited to, N-acetyl-D-galactosamine, N-acetyl-D-glucosamine, and N-acetyl-beta-D-mannosamine. The carbon source can be present in the heterotrophic medium at a concentration of 200 g/L, 175 g/L, 150 g/L, 100 g/L, 60 g/L or less, e.g., at a concentration of 1 to 200 g/L, 5 to 200 g/L, 10 to 200 g/L, 50 to 200 g/L, or 100 to 200 g/L.

The microorganisms can be cultured in medium having a chloride concentration from about 0.5 g/L to about 50.0 g/L (e.g., a chloride concentration from about 0.5 g/L to about 35 g/L, from about 18 g/L to about 35 g/L, or from about 2 g/L to about 35 g/L). The microorganisms described herein can be grown in low chloride conditions, e.g., from about 0.5 g/L to about 20 g/L, or from about 0.5 g/L to about 15 g/L.

The culture medium optionally includes NaCl. The culture medium can include non-chloride-containing sodium salts as a source of sodium. Examples of non-chloride sodium salts suitable for use in accordance with the present methods include, but are not limited to, soda ash (a mixture of sodium carbonate and sodium oxide), sodium carbonate, sodium bicarbonate, sodium sulfate, and mixtures thereof. See, e.g., U.S. Pat. Nos. 5,340,742 and 6,607,900, the entire contents of each of which are incorporated by reference herein. Optionally, the medium comprises 9 g/L chloride when using 20 g/L of carbon, 20 g/L soy peptone, and 5 g/L yeast extract. The medium can comprise 35 g/L chloride when the medium contains 10 g/L carbon, 5 g/L soy peptone, 5 g/L yeast extract and 10 g/L agar. The medium can comprise 2 g/L chloride when the medium contains 20-40 g/L carbon, 1 g/L yeast extract, 1-20 g/L monosodium glutamate (MSG), 0.3-2.0 g/L phosphates, 4 g/L magnesium sulfate, 5-10 g/L ammonium sulfate, 1.5 mL/L trace elements solution, 1 mL/L of vitamin B solution, and 0.1 g/L CaCl₂).

Medium for a microorganism culture can include any of a variety of nitrogen sources. Exemplary nitrogen sources include ammonium solutions (e.g., NH₄ in H₂O), ammonium or amine salts (e.g., (NH₄)₂SO₄, (NH₄)₃PO₄, NH₄NO₃, NH₄OOCH₂CH₃ (NH₄Ac)), peptone, soy peptone, tryptone, yeast extract, malt extract, fish meal, sodium glutamate, soy extract, casamino acids and distiller grains. Concentrations of nitrogen sources in suitable medium typically range between and including about 1 g/L and about 25 g/L (e.g., about 5 to 20 g/L, about 10 to 15 g/L, or about 20 g/L). Optionally, the concentration of nitrogen is about 10 to 15 g/L when yeast extract is the source of complex nitrogen in the medium. Optionally, the concentration of nitrogen is about 1 to 5 g/L when soy peptone is in the medium along with L-Glutamic acid monosodium salt hydrate (MSG) or ammonium sulfate.

The medium optionally includes a phosphate, such as potassium phosphate or sodium-phosphate (e.g., potassium phosphate monobasic).

Inorganic salts and trace nutrients in the medium can include ammonium sulfate, sodium bicarbonate, sodium orthovanadate, potassium chromate, sodium molybdate, selenous acid, nickel sulfate, copper sulfate, zinc sulfate, cobalt chloride, iron chloride, manganese chloride calcium chloride, and EDTA. Optionally, the medium includes at least 1.5 ml/L of a trace element solution. Optionally, the trace element solution comprises 2 mg/mL copper (II) sulfate pentahydrate, 2 mg/mL zinc sulfate heptahydrate, 1 mg/mL cobalt (II) chloride hexahydrate, 1 mg/mL manganese (II) chloride tetrahydrate, 1 mg/mL sodium molybdate dihydrate, and 1 mg/mL nickel (II) sulfate.

The medium can include magnesium sulfate, optionally, with a trace element solution and/or potassium phosphate monobasic.

Vitamins such as pyridoxine hydrochloride, thiamine hydrochloride, calcium pantothenate, p-aminobenzoic acid, riboflavin, nicotinic acid, biotin, folic acid and vitamin B12 can be included in the culture medium.

The pH of the medium can be adjusted to between and including 3.0 and 10.0 using acid or base, where appropriate, and/or using the nitrogen source. Optionally, the medium is sterilized.

Generally, a medium used for culture of a microorganism is a liquid medium. However, the medium used for culture of a microorganism can be a solid medium. In addition to carbon and nitrogen sources as discussed herein, a solid medium can contain one or more components (e.g., agar and/or agarose) that provide structural support and/or allow the medium to be in solid form.

Cultivation of the microorganisms can be carried out using known conditions, for example, those described in International Patent Publication Nos. WO 2007/069078 and WO 2008/129358. For example, cultivation can be carried out for 1 to 30 days (e.g., 1 to 21 days, 1 to 15 days, 1 to 12 days, 1 to 9 days, or 3 to 5 days). Cultivation can be carried out at temperatures between 4 to 30° C. Optionally, cultivation is carried out by aeration-shaking culture, shaking culture, stationary culture, batch culture, fed-batch culture, continuous culture, rolling batch culture, wave culture, or the like. Optionally, cultivation is carried out with a dissolved oxygen content of the culture medium between 1 and 20%, between 1 and 10%, or between 1 and 5%.

The biomass as described herein can be incorporated into a final product (e.g., food or feed supplement, biofuel, etc.). Thus, provided is a method of using the protein-rich biomass. The method optionally includes incorporating the protein-rich biomass into a foodstuff (e.g., a pet food, a livestock feed, or an aquaculture feed).

Oils or lipids can be isolated from the described microorganism culture and used in various food and feed supplements. Suitable food or feed supplements into which the oils can be incorporated include beverages such as milk, water, sports drinks, energy drinks, teas, and juices; confections such as candies, jellies, and biscuits; fat-containing foods and beverages such as dairy products; processed food products such as soft rice (or porridge); infant formulae; breakfast cereals; or the like. Optionally, one or more produced lipids can be incorporated into a dietary supplement, such as, for example, a vitamin or multivitamin. Optionally, an oil produced according to the method described herein can be included in a dietary supplement and optionally can be directly incorporated into a component of food or feed (e.g., a food supplement).

Examples of feedstuffs into which oils or lipids produced by the methods described herein can be incorporated include pet foods such as cat foods; dog foods; feeds for aquarium fish, cultured fish or crustaceans, etc.; or feed for farm-raised animals (including livestock and fish or crustaceans raised in aquaculture). Food or feed material into which the oils or lipids produced according to the methods described herein can be incorporated is preferably palatable to the organism which is the intended recipient. This food or feed material can have any physical properties currently known for a food material (e.g., solid, liquid, soft).

Optionally, one or more of the produced compounds (e.g., PUFAs) can be incorporated into a nutraceutical or pharmaceutical product. Examples of such a nutraceutical or pharmaceutical forms include various types of tablets, capsules, drinkable agents, etc. Optionally, the nutraceutical or pharmaceutical is suitable for topical application (e.g., lotion form). Dosage forms can include, for example, capsules, oils, tablets or the like.

The oil or lipids produced according to the methods described herein can be incorporated into products in combination with any of a variety of other agents. For instance, such compounds can be combined with one or more binders or fillers, chelating agents, pigments, salts, surfactants, moisturizers, viscosity modifiers, thickeners, emollients, fragrances, preservatives, etc., or any combination thereof.

As described herein, a control or standard control refers to a sample, measurement, or value that serves as a reference, usually a known reference, for comparison to a test sample, measurement, or value. For example, a test microorganism, e.g., a microorganism transformed with nucleic acid sequences encoding genes for metabolizing xylose can be compared to a known normal (wild-type) microorganism (e.g., a standard control microorganism). A standard control can also represent an average measurement or value gathered from a population of microorganisms (e.g., standard control microorganisms) that do not grow or grow poorly on xylose as the sole carbon source or that do not have or have minimal levels of xylose isomerase activity, xylulose kinase activity and/or xylose transport activity. One of skill will recognize that standard controls can be designed for assessment of any number of parameters (e.g., RNA levels, polypeptide levels, specific cell types, and the like).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### Example 1. Engineered Microorganisms Comprising Elongases and Desaturases

A C16:0 elongase from Oblongichytrium (Obl) and a Δ9 desaturase from Ulkenia (Ulk) were codon-optimized for ONC-T18 (ATCC Accession No. PTA-6245 also referred to throughout as T18) and placed under the control of the native Δ9 desaturase promoter by cloning the genes upstream of a neomycin resistance (neo-R) resulting in plasmid pHR37 (found in FIG. 2). A 2A and GSG linker were included between each gene for efficient cleavage of the expressed proteins. T18 was biolistically transformed, resulting in G418 resistant transformants, referred to herein as the 95 series.

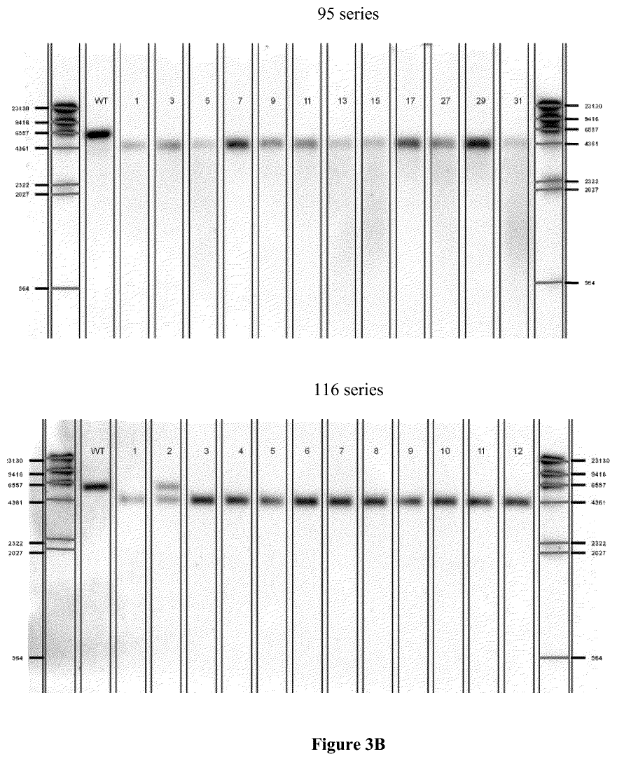

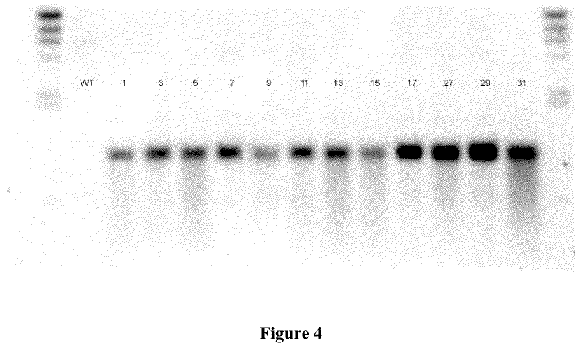

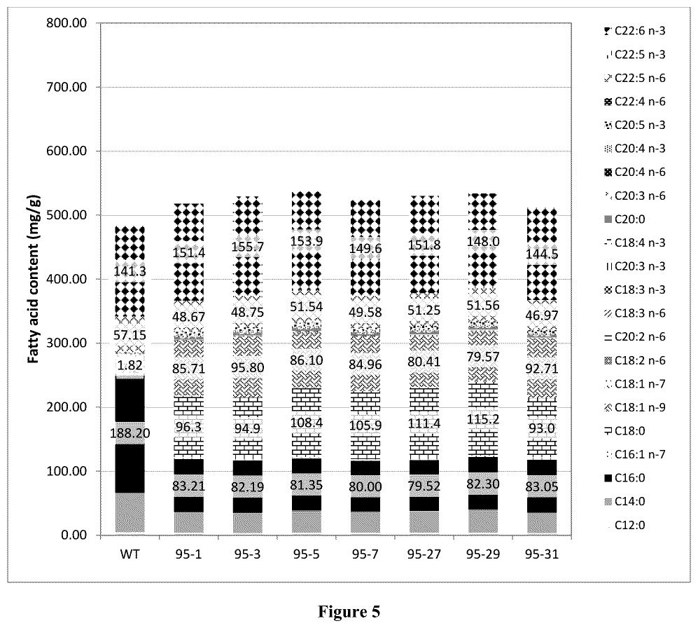

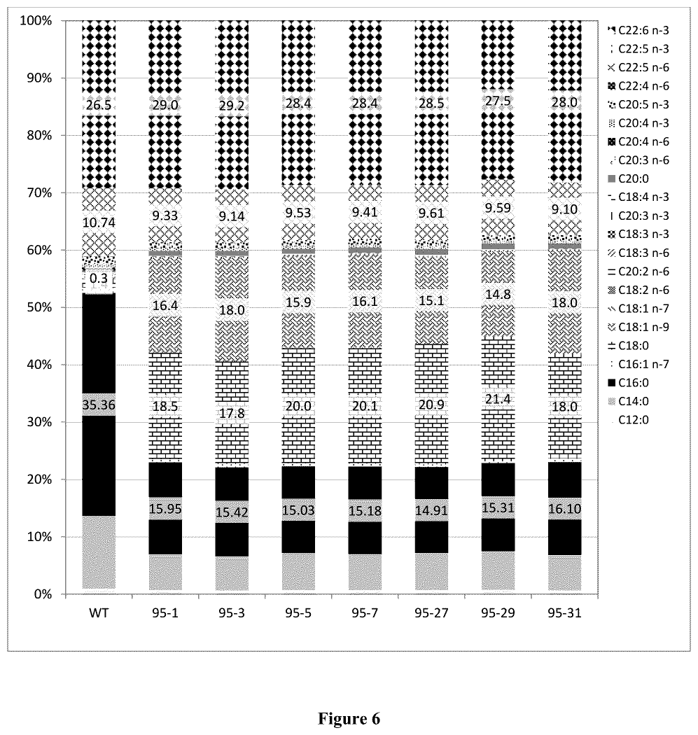

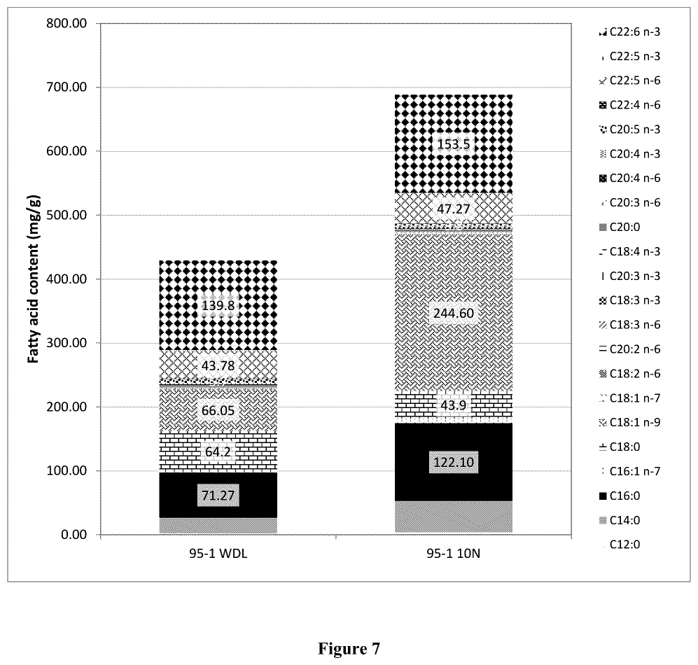

Homozygous homologous recombination at the Δ9 desaturase locus was confirmed by Southern blot using a probe against a region upstream of the Δ9 desaturase promoter and neomycin, as shown in FIGS. 3 and 4. The fatty acid profiles of the 95 series transformants and T18 WT were determined by growing the strains in 25 mL WDL cultures for 7 days, until glucose was depleted (<1 g/L glucose remaining). Harvested biomass was lyophilized and subjected to FAMEs analysis to obtain oil profiles, as shown in FIGS. 5 and 6. T18 WT produced 7.23 milligram C18:0 per gram dried biomass (mg/g) and 1.82 milligram C18:1 oleic acid per gram (mg/g) dried biomass (FIG. 5). The 95 series transformants produced up to 115.15 mg C18:0/g dried biomass (transformant 95-29) and 95.80 mg C18:1 oleic acid/g dried biomass (transformant 95-3). These C18:0/dried biomass and C18: 1/dried biomass ratios represent 1493% and 5164% increases over those found in wildtype (WT) T18, respectively. One 95 series transformant, 95-1, was grown in 25 milliliter (mL) WDL and 25 mL 10% N WDL to compare the oil profile under normal growth and nitrogen stress, respectively. Cultures were grown for 8 days at 25° C. to glucose depletion (<1 g/L glucose remaining). Harvested biomass was lyophilized and subjected to FAMEs analysis to obtain oil profiles, as shown in FIG. 7. There is an increase in C18: 1 oleic acid in low nitrogen to 244.60 mg/g dried biomass from 66.05 mg/g dried biomass, representing a 270.3% increase in oleic acid content in low nitrogen conditions. There is also an increase in total fatty-acids (TFA) in low nitrogen to 695.49 mg/g dried biomass from 434.46 mg/g dried biomass, representing a 60.1% increase.

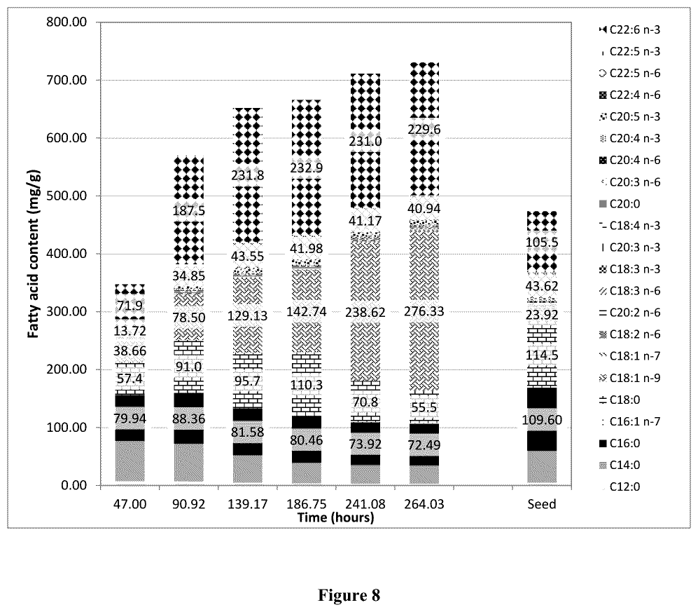

Transformant 95-1 was also grown in a 5 liter (L) fermentation vessel under standard conditions. Briefly, 500 mL Windust Light (with 5 g/L Yeast Extract) seed culture of transformant 95-1 grew for 72 hours at 25° C. Three hundred (300) mL of the culture were used to inoculate 2.7L Windust medium in fermentation vessel (300 g glucose, 6g yeast extract, 9.24g MgSO₄·7H2O, 4.95 g NaCl, 11.7 mg FeCl₃·6H₂O, 24.57 mg citric acid, 7.2 mg CuSO₄·5H₂O, 7.2 mg ZnSO₄·7H₂O, 3.6 mg Na₂MoO₄·2H₂O, 3.6 mg CoCl₂·6H₂O, 3.6 mg MnCl₂·4H₂O, 3.6 mg NiSO₄·6H₂O, 46.26g (NH₄)2SO₄, 5.1g KH₂PO₄, 5.55g K₂HPO₄, 0.3g CaCl₂·2H₂O, 0.09 mg Vitamin B12, 0.09 mg Biotin, 18 mg Thiamine HCl, and 5 drops Biospumex 153K). Glucose feeds of a 750w/v solution were done periodically to maintain a 40-90 g/L glucose concentration within the vessel. Glucose concentration was determined by YSI analyzer (YSI Incorporated, Yellow Springs, Ohio). Consumption rates were maintained at or below 2.5 g glucose/L-h by adjusting stir. Fermentation was considered complete when 1650 g total glucose was consumed. Fermentation was carried out at 20° C. and pH was maintained at 5.75 with addition of 5M NaOH. Vessel operated at 20° C. with 450-650 rpm agitation. Fermentation went on for 264 hours. Samples were taken throughout the fermentation to compare the oil profile at different timepoints, as shown in FIG. 8.

### Example 2. Engineered Microorganisms Comprising Desaturase Promoter

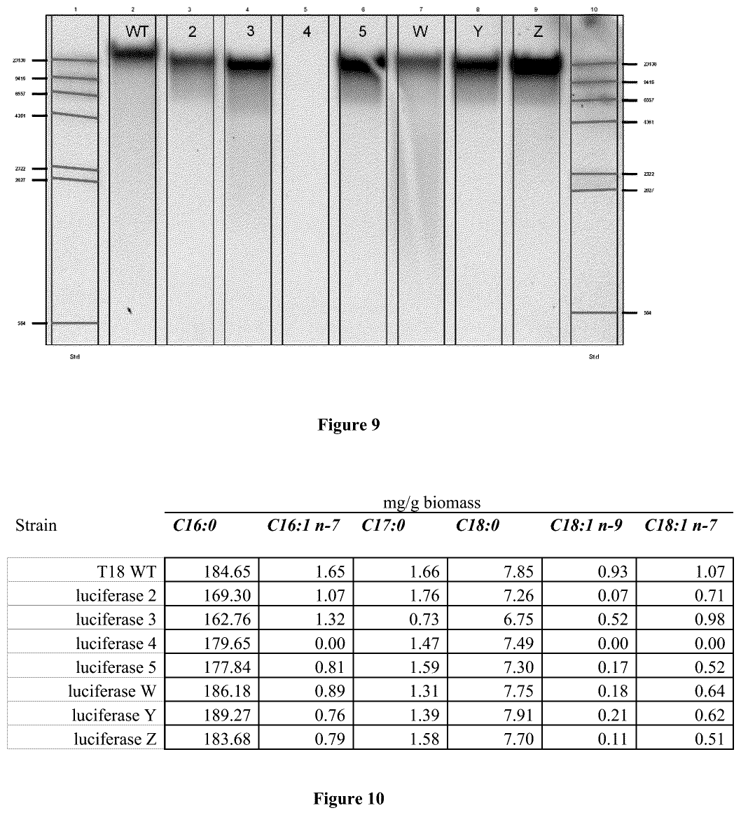

The reporter gene Gaussia luciferase (Gluc) was cloned into an expression construct 3′ to a zeocin resistance gene (ble^{R}), separated by a 2A sequence under control of the native Δ9 desaturase promoter in plasmid pJB12, as shown in FIG. 2. T18 was biolistically transformed with pJB12, resulting in zeomycin-resistant transformants referred to herein as the luciferase series. Southern blot analysis, with a probe for Δ9 desaturase confirmed homozygous homologous recombination at the Δ9 locus in one transformant, as shown in FIG. 9. The luciferase transformants were grown in 50 mL WDL for 5 days. Harvested biomass from these cultures was lyophilized and subjected to FAMEs analysis. FAMEs data in FIG. 10 shows that a homozygous knockout of native Δ9 desaturase in T18 results in the absence of C16:1 n-7, C18:1 n-9 and C18:1 n-7, which is consistent with the suspected function of the gene.

### Example 3. Engineered Microorganisms Comprising Desaturases

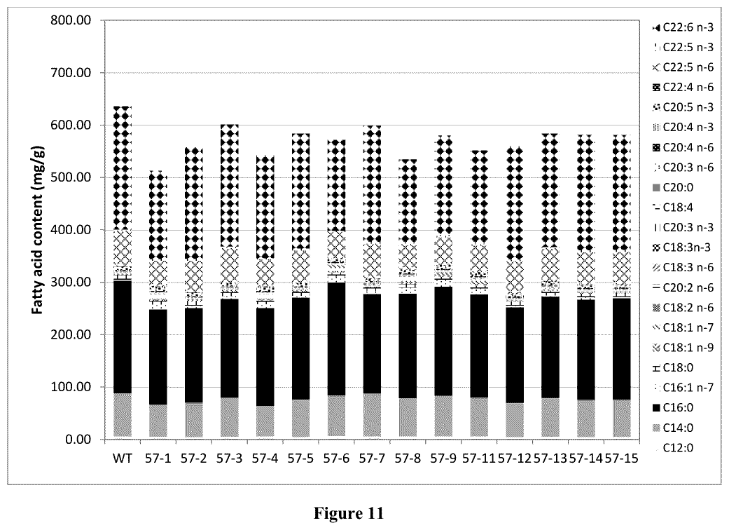

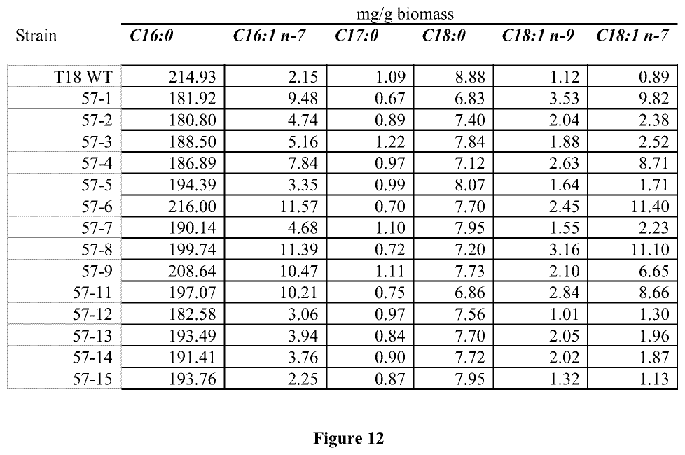

The native T18 Δ9 desaturase was cloned into an expression construct under control of the native T18 α-tubulin promoter resulting in plasmid pJB76, as shown in FIG. 2. T18 was biolistically transformed resulting in zeocin-resistant transformants referred to herein as the 57 series. PCR was used to confirm integration of zeocin-containing construct in transformant strains. The 57 series transformants were grown in 56 mL WDL for 5-7 days, until glucose depletion to <4 g/L. Harvested biomass was lyophilized and subjected to FAMEs analysis, with the resulting FAMEs data shown in FIG. 11. FAMEs data shows increases in C16:1 n-7 and C18:1 n-7 over WT in all transformants, as shown in FIG. 12. Most prominent increase is in transformant 57-6: transformant 57-6 has 11.57 mg C16:1 n-7/g biomass and 11.40 mg C18:1 n-7/g biomass compared to 2.15 mg/g and 0.89 mg/g in WT, respectively, representing increases of 438% and 1180%, respectively, compared to WT.

### Example 4. Engineered Microorganisms Comprising Desaturase Under Control of Native Promoter

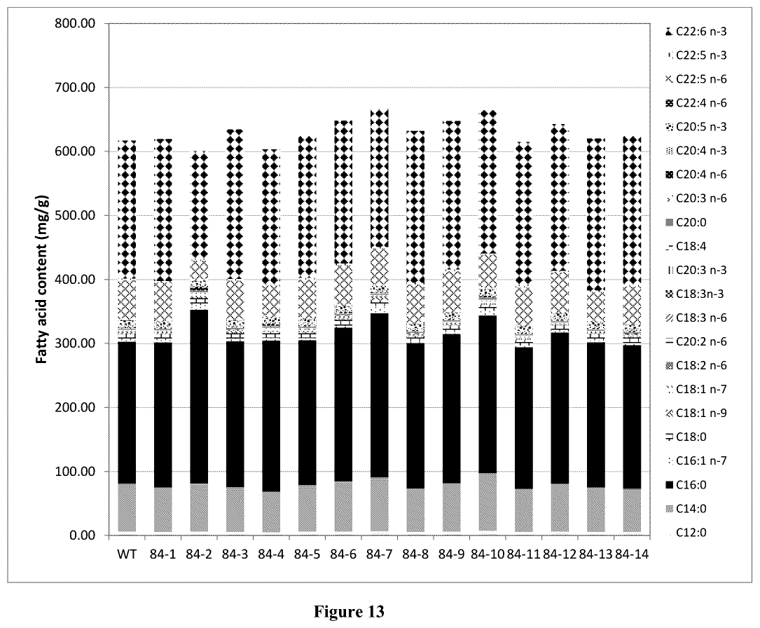

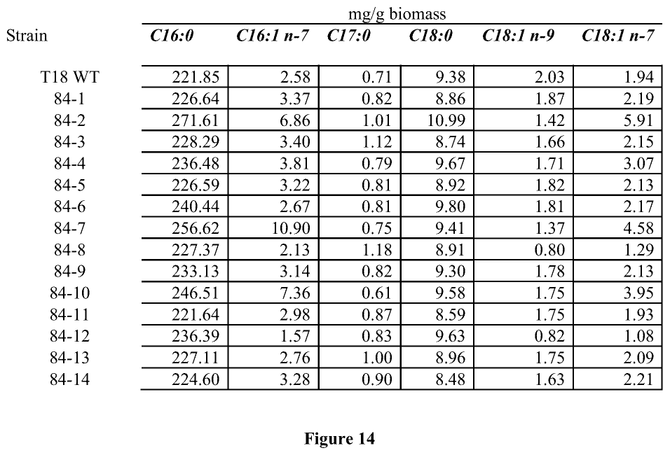

The native T18 49 desaturase was cloned into an expression construct under control of the PUFA synthase subunit B promoter (subB) native to T18 with homology arms to knock out a native fatty-acid elongase resulting in plasmid pJB87, as shown in FIG. 2. Polymerase chain reaction (PCR) analysis was used to confirm integration of zeocin-containing construct in transformant strains. The 84 series transformants were grown in 50 mL WDL for 5-6 days. Harvested biomass was lyophilized and subjected to FAMEs analysis, as shown in FIG. 13. FAMEs data shows an increase in both C16:1 n-7 and C18:1 n-7 over T18 WT in most transformants, as shown in FIG. 14. Most prominent 22-7 increase is in transformant 84-7, which has 10.9 mg C16:1 n-7/g biomass and 4.58 mg C18:1 n-7/g biomass compared to 2.58 mg/g and 1.94 mg/g in WT respectively, representing respective increases of 322% and 136% compared to WT.

### Example 5. Engineered Microorganisms Comprising Δ12 Desaturase

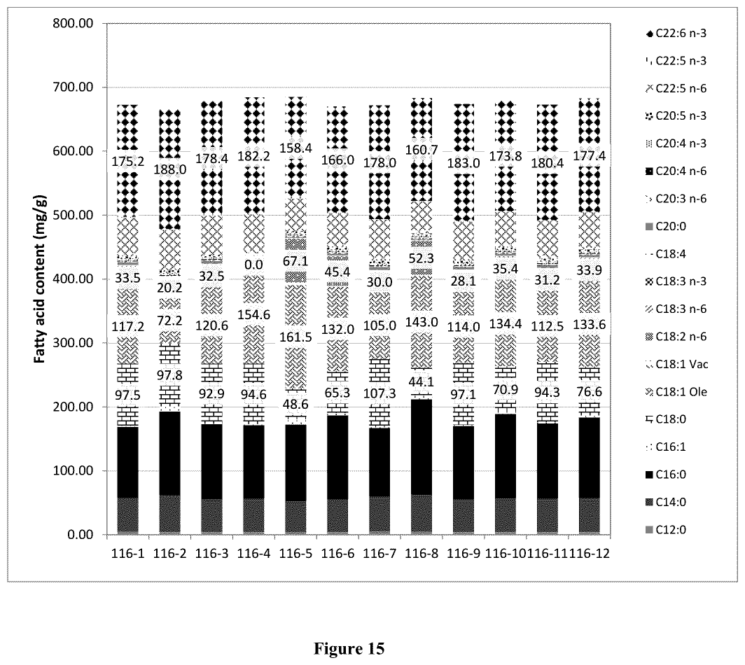

A Δ12 desaturase from Thraustochytrium (Thr) was codon-optimized for T18. This gene was cloned 3′ to C16:0 elongase (Obl), Δ9 desaturase (Ulk) and a zeocin resistance gene (ble^{R}) under the control of the native T18 Δ9 desaturase promoter and separated by 2A sequences resulting in plasmid pHR51, as shown in FIG. 2. T18 was biolistically transformed with pHR51, resulting in zeomycin resistant transformants, called 116 series. Southern blot analysis with a probe against a region upstream of the Δ9 desaturase promoter confirmed either homozygous or heterozygous homologous recombination at the Δ9 desaturase locus in transformants, as shown in FIG. 3. Eleven out of twelve transformants were double knockouts, where the 116-2 transformant is heterozygous for homologous recombination at Δ9 locus. Fatty acid profiles of the 116 series transformants and T18B WT grown in 10% N WDL and harvested at glucose depletion were determined by FAMEs analysis, as shown in FIG. 15. T18 WT contains no C18:2 linoleic acid while the 116 series transformants produce up to 67.1 mg/g dried biomass, which represents 9.7% weight percent of the total fatty acids. Weight percent determined by mg/g specific fatty acid over mg/g total fatty acids.

### Example 6. Engineered Microorganisms Comprising Δ12 Desaturase Under Control of Tubulin Promoter

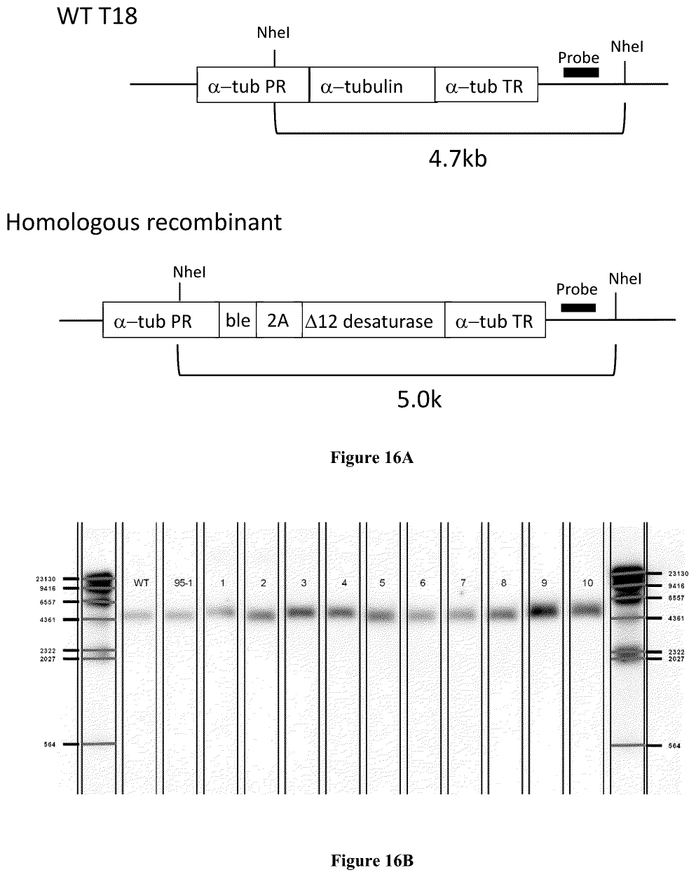

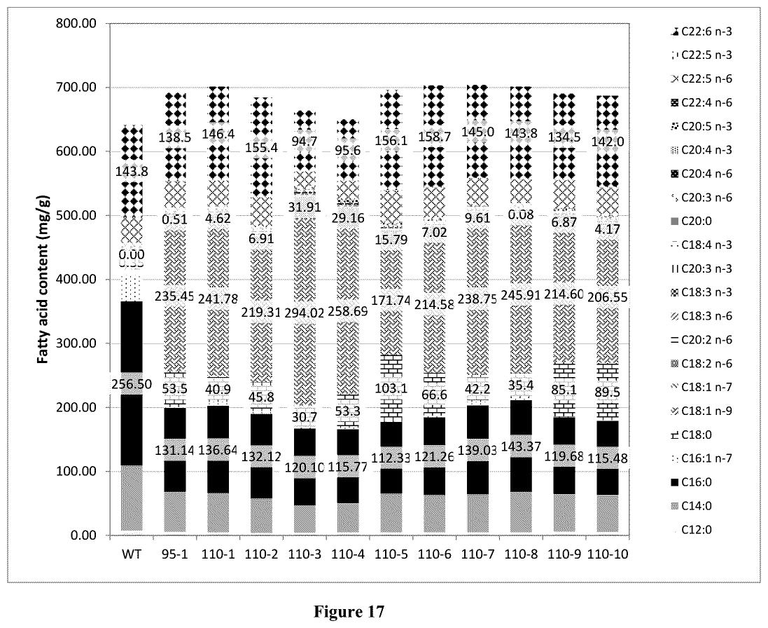

The Δ12 desaturase (Thr) was cloned under control of the alpha-tubulin promoter native to T18 resulting in plasmid pHR47, as shown in FIG. 2. The T18 transformant 95-1 (the oleic acid-producer shown in FIG. 5) was biolistically transformed with pHR47 resulting in zeocin-resistant transformants, referred to herein as the 110 series transformants. Southern blotting indicated possible alpha-tubulin knockouts: 110-1, 3, 4, 9 and 10 transformants, as shown in FIG. 16. The 110 series transformants were grown in 25 mL 10% N WDL for 6 days until glucose was depleted to <10 g/L remaining. Harvested biomass was lyophilized and subjected to FAMEs analysis to obtain oil profiles, as shown in FIG. 17. T18 WT contains no 18:2 linoleic acid, parent 95-1 contains 0.51 mg linoleic acid/g dried biomass and the 110-3 transformant produces 31.91 mg linoleic acid/g dried biomass which represents 4.8% weight percent of the total fatty acids and a 6257% increase over the 95-1 parent.

### Example 7. Engineered Microorganisms Comprising Subunits of PUFA Synthase

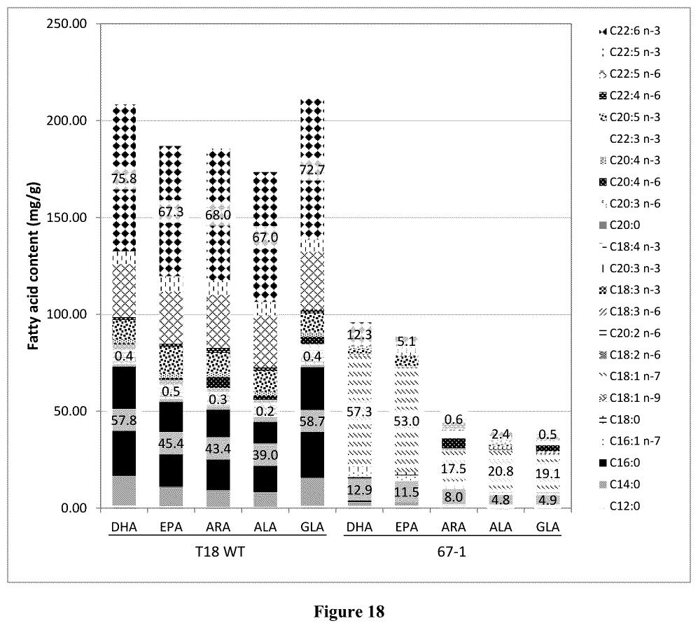

Two protein domains from Shewanella PfaC (a subunit of a PUFA synthase) are codon optimized for T18 and cloned under control of the subB promoter in plasmid pHR26. T18 was biolistically transformed with plasmid pHR26 as described in FIG. 2. Since using homology arms from native T18 PUFA synthase subunit B may result in homologous recombination at subB and a knockout of subB, transformants were recovered on medium supplemented with 0.5 millimolar (mM) DHA. One zeocin-resistant transformant was obtained, referred to herein as the 67-1 transformant. When streaked onto plate lacking PUFA supplementation, the 67-1 transformant could not grow, indicating that transformant 67-1 was auxotrophic. WT T18 and transformant 67-1 were grown in 50 mL WDL cultures, supplemented at 0.5 mM with different free fatty-acid PUFAs in each culture: DHA, EPA, ARA, ALA and GLA. Cultures grew for 14 days, at which time T18 cultures had completely consumed glucose (<1 g/L remaining) and 67-1 cultures were left with 20.6 g/L glucose (DHA culture), 28.1 g/L glucose (EPA culture), 37.9 g/L glucose (ARA culture), 54.9 g/L glucose (ALA culture), and 54.6 g/L glucose (GLA culture), respectively. Harvested biomass was lyophilized and subjected to FAMEs analysis to obtain oil profiles, as shown in FIG. 18. The average 18:1 n-7 content in WT T18 for these cultures grown in the presence of the different free fatty-acid PUFAs was 0.346 mg/g dried biomass. The average 18:1 n-7 content in the 67-1 transformant for these cultures grown in the presence of the different free fatty-acid PUFAs was 33.54 mg/g dried biomass, representing a 9694% increase over WT T18. Conversion of some of the cultures fed PUFAs indicates that the presence of limited fatty-acid elongases and desaturases in WT T18. Notably, the 67-1 cultures fed ALA accumulated EPA (3.88 mg/g or 8.8% weight percent of the total fatty acids) and the 67-1 cultures fed GLA accumulates ARA (3.63 mg/g or 8.5% weight percent of the total fatty acids).

### Example 8. Engineered Microorganisms Comprising Ω-3 Desaturase

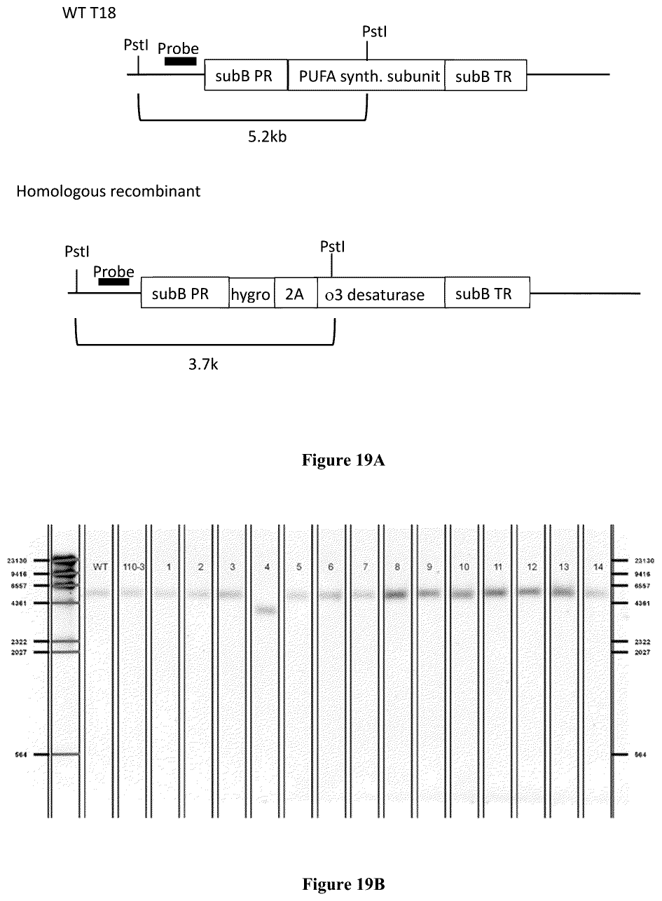

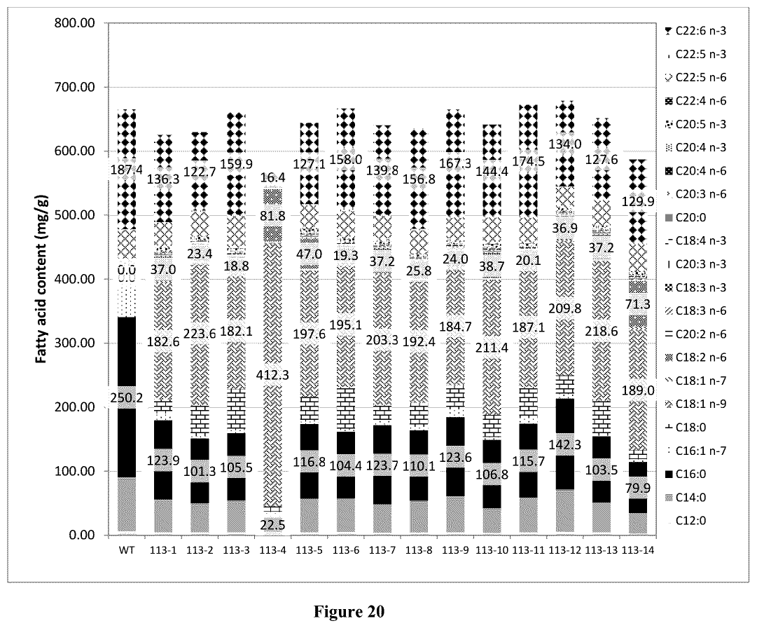

An Ω-3 desaturase from Oblongichytrium (Obl) is codon optimized for T18 and cloned into an expression construct under control of the PUFA synthase subunit B promoter (subB) native to T18 in plasmid pHR52, shown in FIG. 2. T18 transformant 110-3 (the linoleic acid-producer shown in FIG. 17) was biolistically transformed with pHR52 resulting in hygromycin-resistant transformants referred to herein as the 113 series transformants. Knocking out subunit B of PUFA synthase incapacitates PUFA synthase and results in auxotrophs requiring PUFA-supplementation (as described in transformant 67-1). For this reason, transformants are recovered on medium supplemented with 0.5 mM DHA. One auxotrophic transformant was recovered, referred to herein as the 113-4 transformant. It was confirmed by Southern blot that 113-4 is a homozygous knockout at subunit B of PUFA synthase, shown in FIG. 19. The 113 series transformants were grown in 25 mL 10% N WDL in a flask (culture for 113-4 also contained 0.5 mM DHA) for 7 days until glucose was depleted to <1 g/L. Transformants 113-4 and 113-14 still had considerable glucose at day 7 and were allowed to grow for two additional days, at which time the 113-4 culture had 14.2 g/L glucose and 113-14 culture had 11.8 g/L glucose. All cultures were harvested for biomass, which was lyophilized and subjected to FAMEs analysis to obtain oil profiles, shown in FIG. 20.

### Example 9. Engineered Microorganisms Comprising Ω-3 Desaturase Under Control of Native Promoter

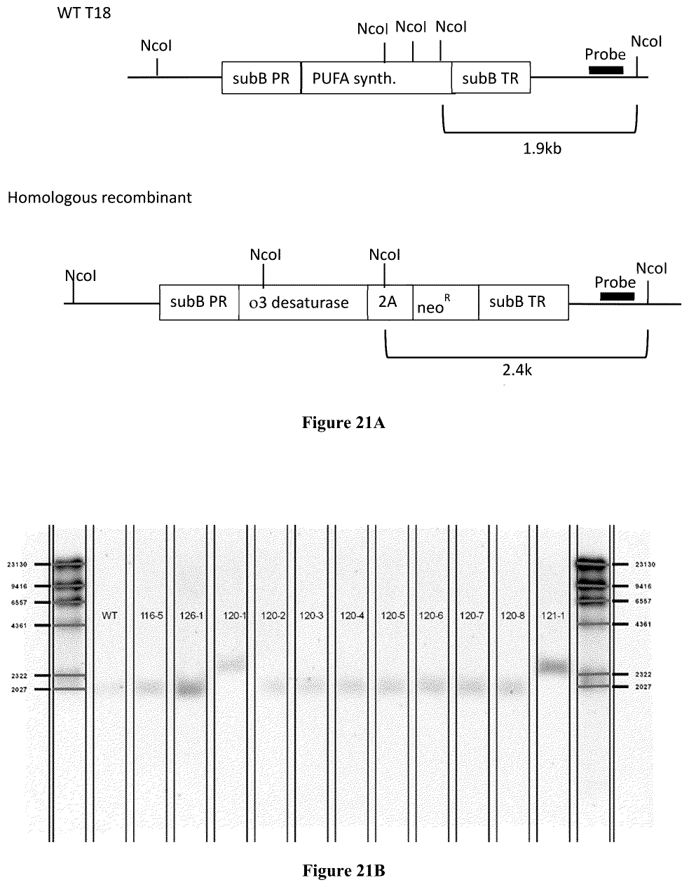

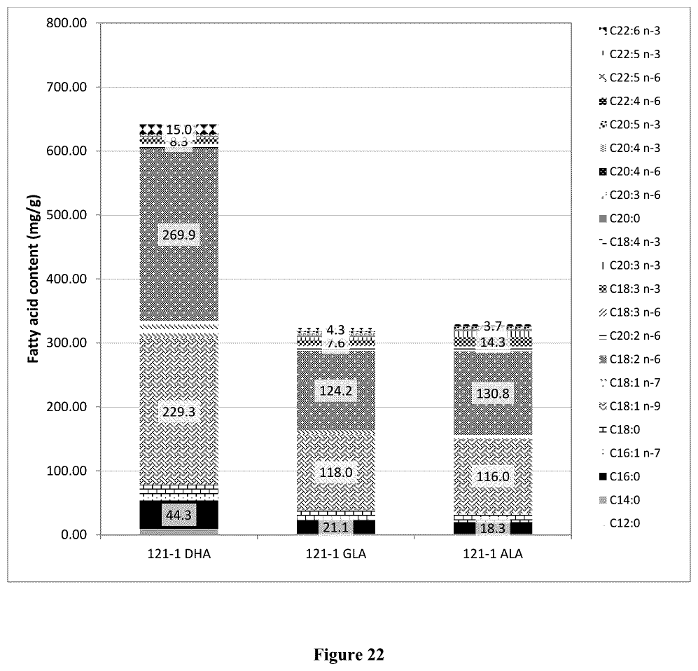

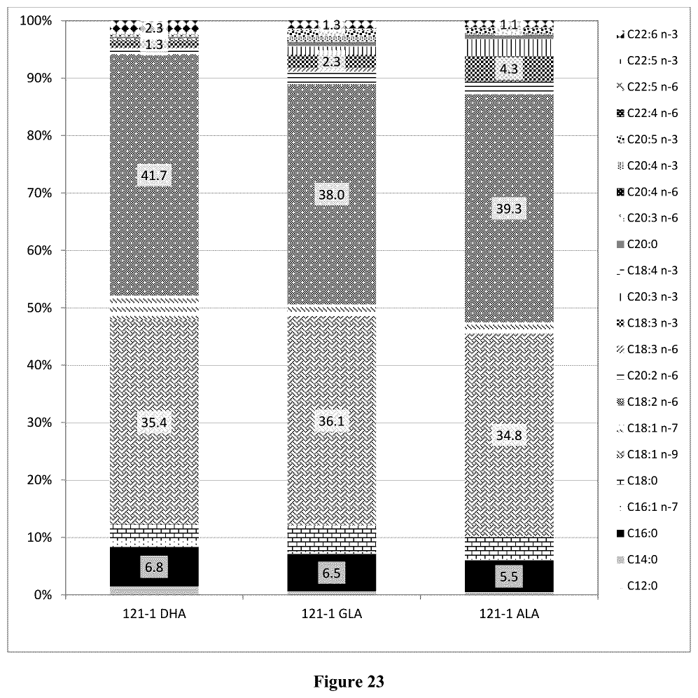

An Ω-3 desaturase (Obl) under control of the subB promoter native to T18 is cloned 5′ to a neomycin-resistance marker in pHR58, shown in FIG. 2 and biolistically transformed into strain 116-5. One G418-resistant transformant was recovered on medium supplemented with 0.5 mM DHA, that transformant referred to herein as 121-1. It was confirmed by Southern Blot that 121-1 is a homozygous knockout at subunit B of PUFA synthase, shown in FIG. 21. Transformant 121-1 was grown in 25 mL 0.5 mM DHA 10% N WDL, as well as in 25 mL WDL cultures containing 0.5 mM GLA or 0.5 mM ALA. The DHA-supplemented culture was brought to glucose depletion in 7 days. The GLA- and ALA-supplemented cultures grew for 8 days until the 121-1 transformant in GLA had 16.4 g/L glucose remaining and the 121-1 transformant in ALA had 21.6 g/L glucose remaining. All cultures were harvested for biomass which was lyophilized and subjected to FAMEs analysis to obtain oil profiles, shown in FIGS. 22 and 23.

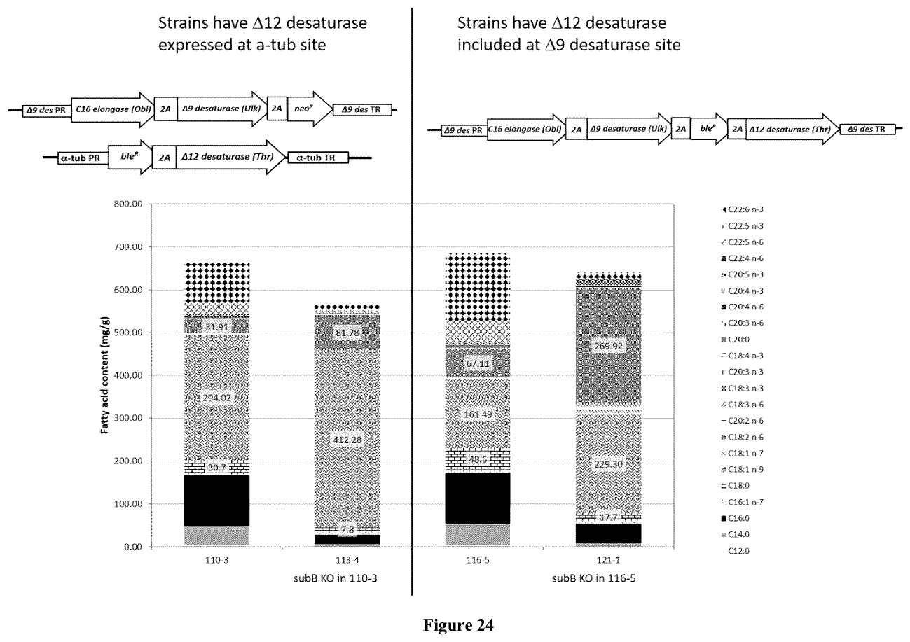

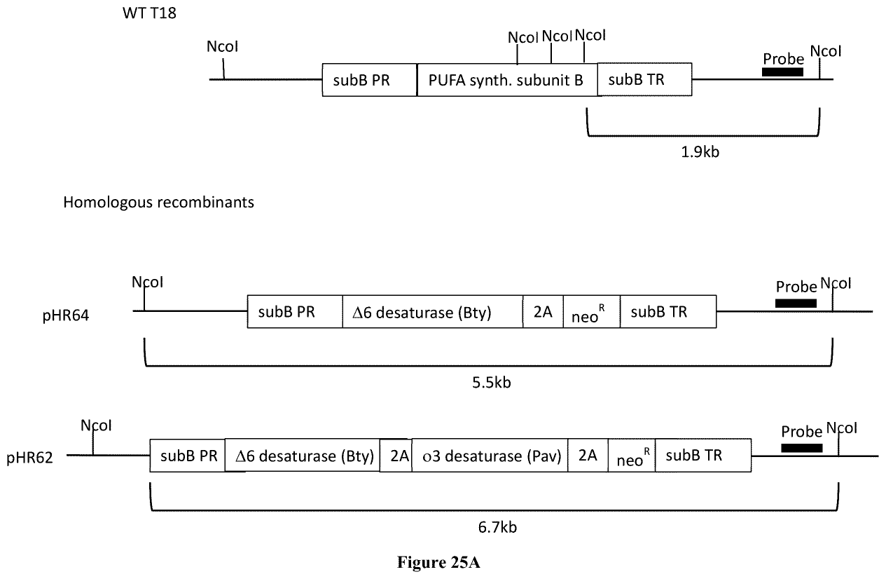

An unexpected aspect of the invention is the finding that expressing a gene in tandem with other genes at the native Δ9 desaturase site leads to more efficient substrate conversion than if the gene is expressed at a discrete site. Transformation series 110 expresses Δ12 desaturase (Thr) in a parent with both a C16:0 elongase (Obl) and a D9 desaturase (Ulk) expressed at the native T18 Δ9 desaturase site. Transformant 110-3 produces 31.91 mg 18:2-6/g biomass. When PUFA synthase subB is knocked out in strain 110-3, the resulting strain produces up to 81.78 mg 18:2-6/g biomass. Transformation series 116 expresses Δ12 desaturase (Thr) together on the same open-reading frame as C16 elongase and Δ9 desaturase at the native T18 Δ9 desaturase site. Transformant 116-5 produces 67.11 mg 18:2-6/g biomass. When PUFA synthase subB is knocked out in strain 116-5, the resulting strain produces up to 269.92 mg 18:2-6/g biomass. This represents a 330% increase in 18:2-6, which is the product of the Δ12 desaturase. These results are shown in FIG. 24.

### Example 10. Engineered Microorganisms Comprising Δ6 Desaturase

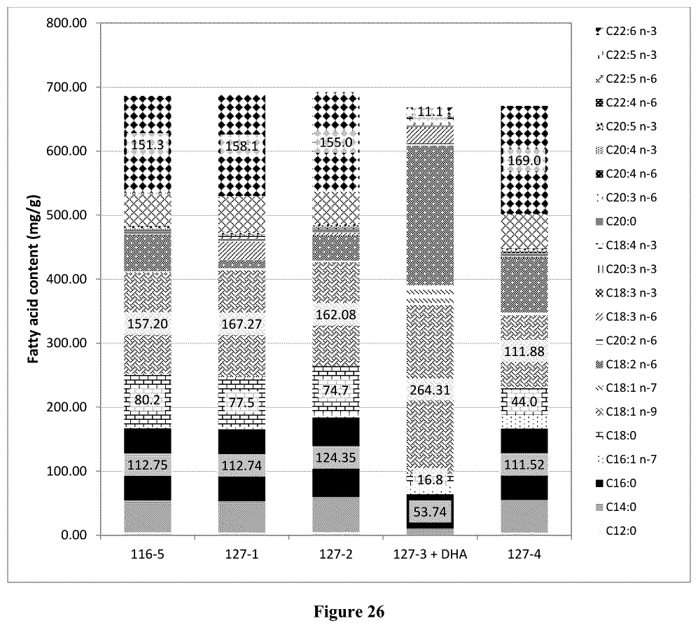

A Δ6 desaturase from Botryochytrium (Bty) was codon-optimized for T18 and cloned into an expression construct under control of the subB promoter in pHR64, shown in FIG. 2. Transformant 116-5, which produces linoleic acid (LA, C18:2 n-6), was biolistically transformed with pHR64. Knocking out subunit B of PUFA synthase generally results in auxotrophic strains, so transformants were recovered on medium supplemented with 0.5 mM DHA. Four G418-resistant transformants were recovered. It was confirmed by Southern blot that 127-3 is a homozygous knockout at subunit B of PUFA synthase, shown in FIG. 24. The 127 series of transformants was grown in 25 mL 10% N WDL (0.5 mM DHA was added to the 127-3 culture) for 6-7 days until glucose depletion (<1 g/L glucose remaining). All cultures were harvested for biomass, which was lyophilized and subjected to FAMES analysis to obtain oil profiles, shown in FIG. 26.

### Example 11. Engineered Microorganisms Comprising Δ6 Desaturase and Ω-3 Desaturase

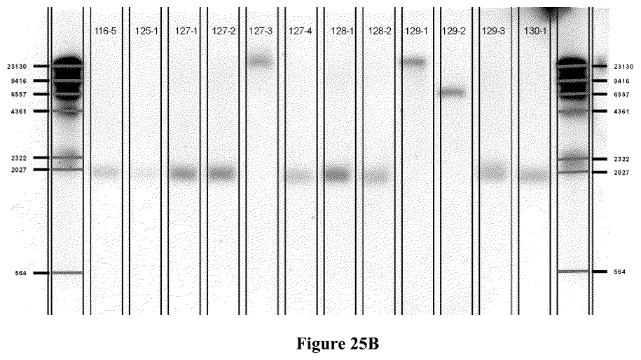

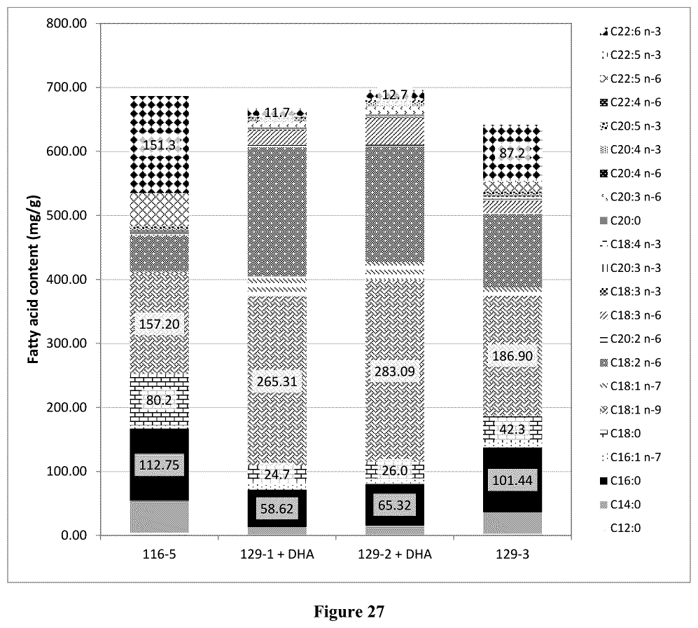

A Δ6 desaturase (Bty) and an Ω-3 desaturase from Pavlova pinguis (Pav) were codon-optimized for T18 and cloned into an expression vector under control of the subB promoter in pHR62, shown in FIG. 2. Strain 116-5, which produces linoleic acid (LA), was biolistically transformed with pHR62. Transformants were recovered on medium supplemented with 0.5 mM DHA. Three G418-resistant transformants were recovered. It was confirmed by Southern blot that 129-1 and 129-2 were homozygous knockouts at subunit B of PUFA synthase, shown in FIG. 25. The 129 series of transformants was grown in 25 mL 10% N WDL (0.5 mM DHA was added to the 129-1 and 129-2 cultures) for 6-7 days until glucose depletion (<1 g/L glucose remaining). All cultures were harvested for biomass which was lyophilized and subjected to FAMEs analysis to obtain oil profiles, shown in FIG. 27.

### Example 12. Engineered Microorganisms Comprising Knockout of SubB

PUFA auxotrophic strains created through knockout of subB in transformant 116-5 were recovered to PUFA prototrophy. Reversion of auxotrophy was achieved by passaging cells from from various modified strains in decreasing concentrations of DHA, beginning with 0.5 mM DHA, passaging into 0.25 mM DHA, then 0.05 mM DHA and finally WDL medium unsupplemented with PUFA.

After passaging, axenic strains able to grow without PUFA supplementation were isolated. Transformant 121-1, which expresses Ω-3 desaturase (Obl) from the subB locus, was inoculated in 10 mL WDL+0.5 mM DHA and allowed to grow for 7 days. A 100 μL sample of the 0.5 mM DHA culture was used to inoculate 10 mL WDL+0.25 mM DHA culture and allowed to grow for 7 days. A 100 μL sample of 0.25 mM DHA culture was used to inoculate 10 mL WDL+0.05 mM DHA culture and allowed to grow for 7 days. A 100 μL sample of 0.05 mM DHA culture was spread on Windust plate and allowed to grow for 6 days. A few colonies that grew after 6 days were restreaked on a second Windust plate and allowed to grow for 12 more days. A mixed population was restreaked onto a third Windust plate allowed to grow for three more days. Two 10 mL WDL cultures were inoculated with single colonies from the third WD plate and allowed to grow for 4 or 7 more days, referred to herein as 121-1-S and 121-1-F, respectively. A 100 μL sample of the 4 or 7 day 121-1-S and 121-1-F cultures were used to inoculate 10 mL WDL cultures, which were then allowed to grow for 3 days. These 3-day WDL cultures that had been inoculated with 121-1-S and 121-1-F, respectively, were used to make frozen stocks and 100 μL samples of each stock was used to inoculate 25 mL WDL and 10% N WDL FAMEs cultures of both 121-1-S and 121-1-F. These WDL FAMEs cultures grew for 8 days until 121-1-S had 2.57 g/L glucose and 121-1-F grew for 11 days until 121-1-S had 12.9 g/L glucose and 121-1-F had 12.9 g/L glucose.

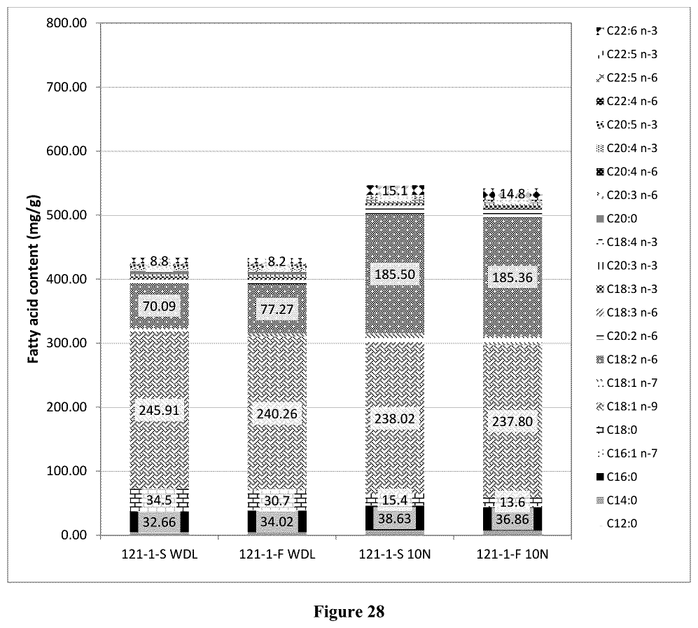

Cultures were harvested for biomass which was lyophilized and subjected to FAMEs analysis to obtain oil profiles, shown in FIG. 28. Transformants 127-3 and 129-2, which express Δ6 desaturase (Bty) (127-3) or both Bty and an (23 desaturase (Pav) (129-2) from the subB locus, were also initially auxotrophic and recovered to prototrophy. Ten milliliter (10 mL) samples of WDL+0.5 mM DHA were inoculated with 127-3 or 129-2 from plate and allowed to grow for 3 days. A 50 μL sample of 0.5 mM DHA cultures was used to inoculate 10 mL WDL+0.25 mM DHA cultures. After 3 days, the 0.25 mM DHA cultures were moved from 25° C. to 20° C. After allowing the 0.25 mM DHA cultures to grow for 6 days total, 100 μL samples of the DHA cultures were used to inoculate 10 mL WDL+0.05 mM DHA cultures, and those cultures were allowed to grow for 7 days.

A 100 μL sample of transformant 129-2 0.05 mM DHA culture (first culture) was used to inoculate a second culture (10 mL WDL+0.05 mM DHA) and allowed to grow for 7 days. A 100 μL sample of the second transformant 129-2 0.05 mM DHA culture was used to inoculate a third 10 mL WDL culture. The third 10 mL WDL (unsupplemented) 129-2 culture was allowed to grow for 6 days, at which time a 100 μL sample was used to inoculate a fourth 10 mL WDL culture. The fourth 129-2 WDL culture grew for 8 days.

A 100 μL sample of transformant 127-3 0.05 mM DHA culture grown for 7 days was used to inoculate 10 mL WDL (unsupplemented) and allowed to grow for 13 days. A 100 μL sample of the 127-3 WDL culture was used to inoculate a second 10 mL WDL culture and allowed to grow for 8 days.

After the 129-2 WDL(2P) and 127-3 WDL(2P) cultures were allowed to grow at 25° C. for 8 days, a 100 μL sample of each culture was used to inoculate 10 mL WDL(3P) for 129-2 and 127-3. The 129-2 WDL(3P) and 127-3 WDL(3P) cultures were allowed to grow for 3 days, after which frozen stocks were made of each. Mixed populations of prototrophic 129-2 and 127-3 were revived from glycerol stocks on Windust plates and streaked for single colonies onto a separate Windust plate. Colonies for 127-3 grew in 6 days, where four of these colonies were picked and designated 127-3-T,W,R+P. The colonies for 129-2 grew in 10 days, after which four of these colonies were picked and designated 129-2-T,W,R+P. A 100 μL sample of 3 day-old 10 mL WDL axenic strains was used to make frozen stocks and inoculate 25 mL 10% N WDL cultures. The 127-3-T,W,R+P cultures grew for 8 days until all glucose was depleted. The 129-2-T,W,R+P cultures grew for 18 days. Remaining glucose concentrations were 8.44, 9.61, 22.2 or 10.5 g/L glucose in the 129-2-T, 129-2-W, 129-2-R and 129-2-P cultures, respectively.

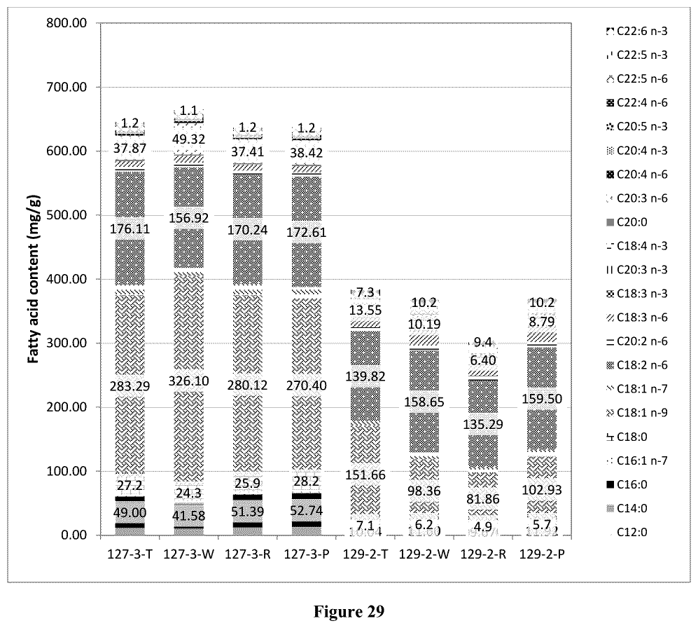

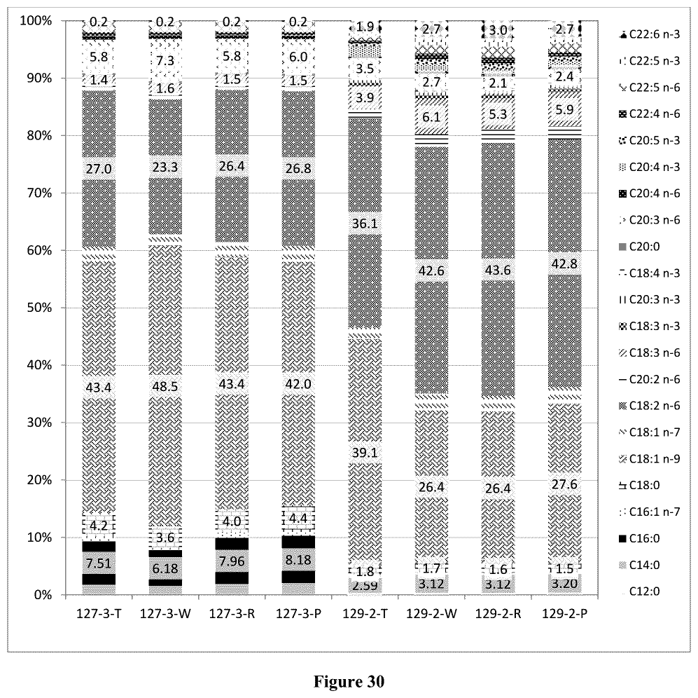

Cultures were harvested for biomass which was lyophilized and subjected to FAMEs analysis to obtain oil profiles, shown in FIGS. 29 and 30. The 127-3 axenic transformants produce up to 49.32 mg C20:3(n-6) (DGLA)/g dried biomass, representing 7.3% of the FAMEs profile up from 0.68 mg/g DGLA in 116-5 or 0.1% of the FAMEs profile.

### Example 13. Engineered Microorganisms Comprising Multiple Desaturases

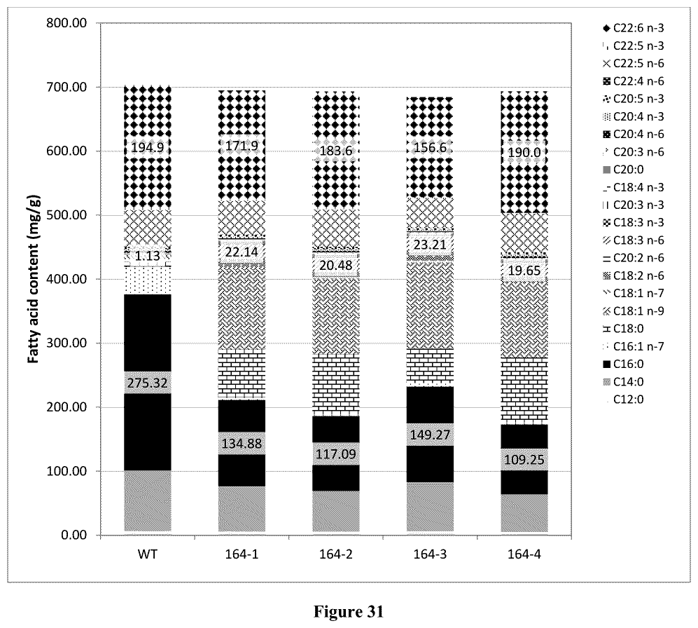

A Δ6 desaturase (Bty) was cloned 3′ to C16:0 elongase (Obl), Δ9 desaturase (Ulk) and a zeocin resistance gene (ble^{R}) and 5′ to A12 desaturase (Thr) under the control of the native T18 Δ9 desaturase promoter and separated by 2A sequences resulting in plasmid pHR84, shown in FIG. 2. T18 was biolistically transformed with pHR84 resulting in zeomycin resistant transformants, referred to herein as the 164 series. Fatty acid profiles of the 164 series transformants and T18B WT grown in 10% N WDL and harvested at glucose depletion were determined by FAMEs analysis, shown in FIG. 31. T18 WT contained 1.13 mg C18:3-6 (γ-linoleic acid)/g dried biomass while 164 series transformants produce up to 23.2 mg/g, which represents 3.4% weight percent of the total fatty acids.

### Example 14. Engineered Microorganisms Comprising Multiple Desaturases Under Control of Native Promoter

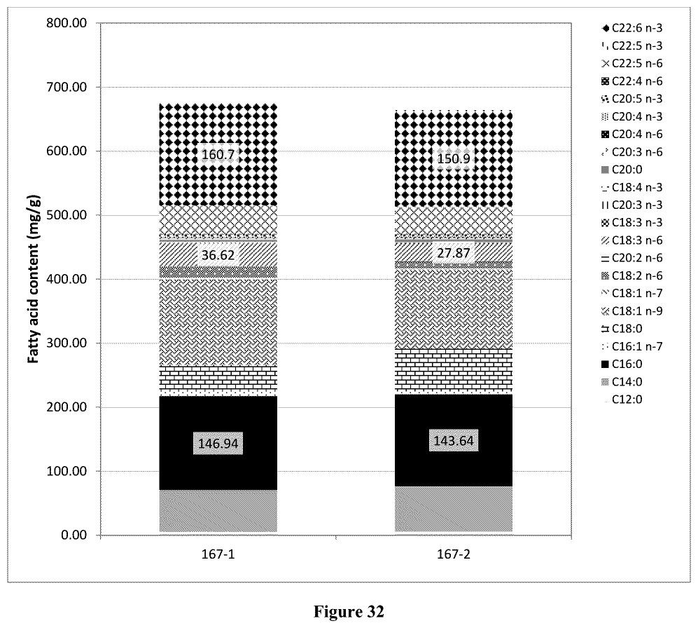

Ω-3 desaturase (Pav) was cloned 3′ to C16:0 elongase (Obl) and 5′ to Δ9 desaturase (Ulk), a zeocin resistance gene (ble^{R}), Δ6 desaturase (Bty) and Δ12 desaturase (Thr) under the control of the native T18 Δ9 desaturase promoter and separated by 2A sequences resulting in plasmid pHR86, shown in FIG. 2. T18 was biolistically transformed with pHR86 resulting in zeomycin resistant transformants called 167 series. Fatty acid profiles of the 167 series strains grown in 10% N WDL and harvested at glucose depletion were determined by FAMEs analysis, shown in FIG. 32. 167 series transformants produce up to 36.6 mg GLA/g dried biomass which represents 5.3% weight percent of the total fatty acids.

### Example 15. Engineered Microorganisms Comprising Desaturases Under Control of Tubulin Promoter

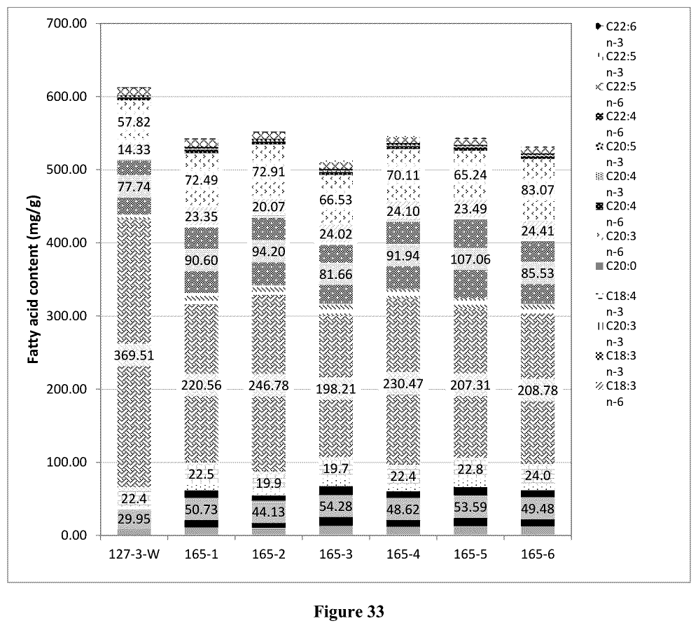

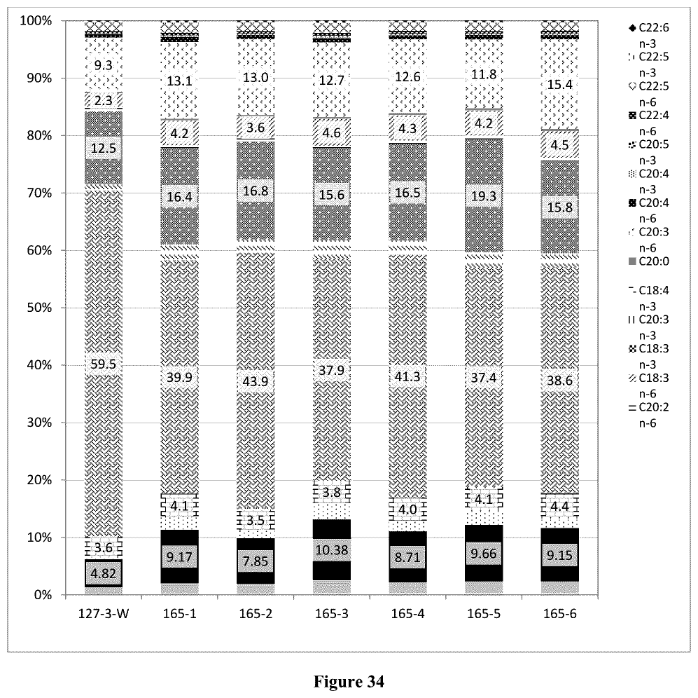

A Δ5 desaturase (Thr) was codon-optimized for T18 and cloned 3′ to a hygromycin resistance gene (Hygro) into an expression vector containing the alpha-tubulin promoter native to T18 and the plasmid was called pHR83, shown in FIG. 2. Strain 127-3-W, which produced DGLA, was biolistically transformed with pHR83. Six hygromycin-resistant transformants were recovered. Fatty-acid profiles of the 165 series transformants grown with parent 127-3-W in 10% N WDL and harvested at or near glucose depletion (7.04, 2.21 and 8.06 g/L glucose remained in 165-3, 165-5 and 165-6, respectively) were determined by FAMEs analysis, shown in FIGS. 33 and 34. The 165 series transformants produced up to 83.07 mg DGLA/g dried biomass which represents 15.4% weight percent of the total fatty acids.

### Example 16. Engineered Microorganisms Comprising Delta 5 Desaturase

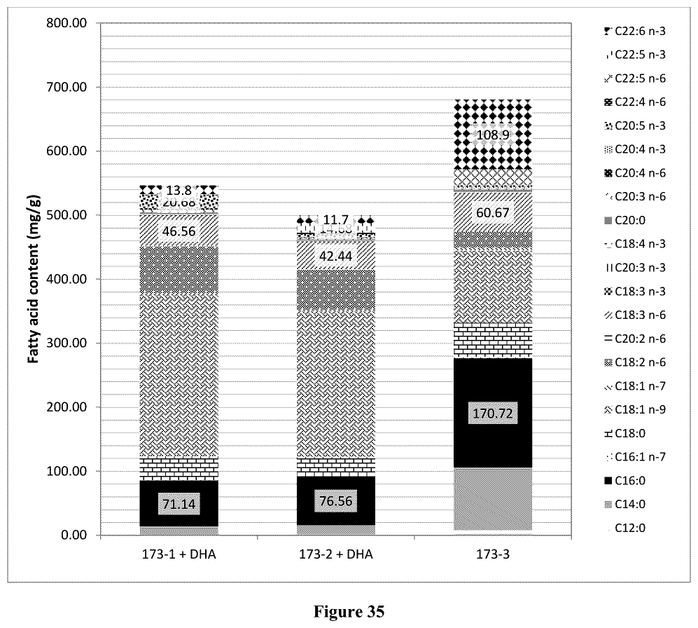

A Δ5 desaturase (Thr) was cloned 5′ to a G418 resistance gene (neo) into an expression vector under control of the subB promoter in pHR95, shown in FIG. 2. Strain 167-1, which produces GLA, was biolistically transformed with pHR95. Transformants were recovered on medium supplemented with 0.75 mM DHA. Three G418-resistant transformants were recovered. Transformants 173-1 and 173-2 were also DHA auxotrophs. 173-1 and 173-2 were grown in 25 mL 10% N WDL+0.5 mM DHA for 7 days until glucose consumption stalled (˜13 g/L glucose remaining). Transformant 173-3 was grown in 10% N WDL for 4 days until glucose depletion (<1 g/L glucose remaining). All cultures were harvested for biomass which was lyophilized and subjected to FAMEs analysis to obtain oil profiles, shown in FIG. 35.

### Example 17. PUFA Auxotrophic Engineered Microorganisms Recovered to PUFA Prototrophy

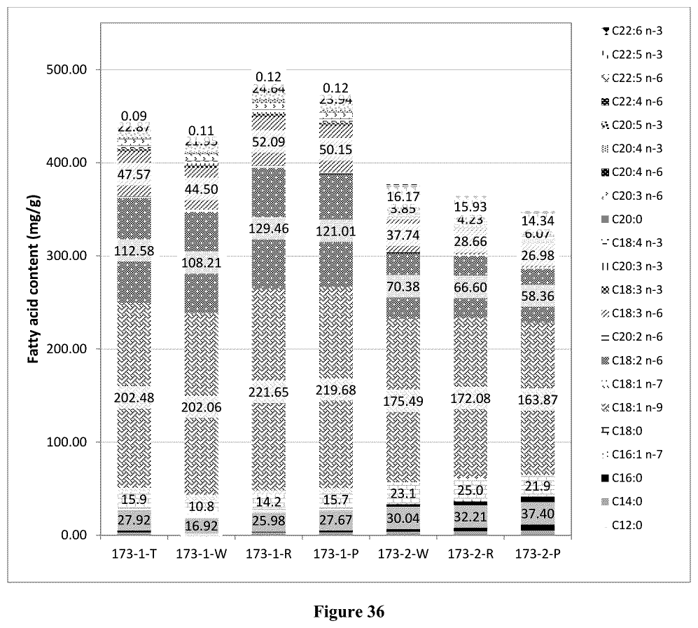

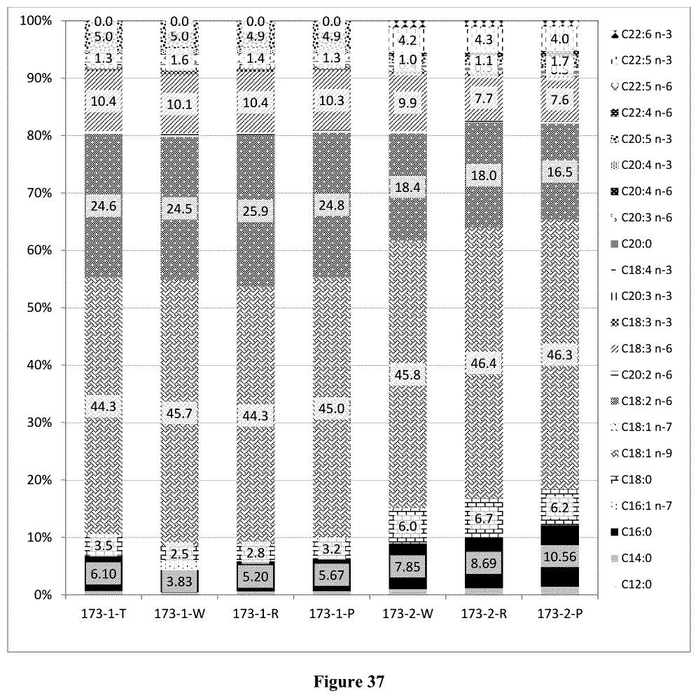

PUFA auxotrophic strains created through knockout of subB in strain 167-1 were recovered to PUFA prototrophy. Reversion of auxotrophy was achieved by passaging cells from 173-1 and 173-2 in decreasing concentrations of DHA, beginning with 0.5 mM DHA, passaging into 0.25 mM DHA, 0.125 mM DHA, 0.05 mM DHA and finally WDL medium unsupplemented with PUFA. Mixed populations of prototrophic 173-1 and 173-2 were revived on Windust plates and streaked for single colonies onto a separate Windust plate. Colonies for 173-1 grew in 6 days, and four were picked and designated 173-1-T, 173-1-W, 173-1-R and 173-1-P. Colonies for 173-2 grew in 7 days, and four were picked and designated 173-2-T, 173-2-W, 173-2-R, and 173-2-P. 100 μL of 3 day-old 10 mL WDL axenic strains was used to make frozen stocks and inoculate 25 mL 10% N WDL cultures. The 173-1-T, 171-1-W, 171-1-R, and 171-1-P cultures grew for 7-10 days until glucose consumption stalled. 173-2-W, 173-2-R, and 173-2-P cultures grew for 7 days. One strain, 173-2-T, did not grow well in culture. Remaining glucose concentrations were 24.8, 26.8, 21.4, 24.5, 23.7, 32.1 and 25.6 g/L glucose in 173-1-T, 173-1-W, 173-1-R, 173-1-P, 173-2-W, 173-2-R and 173-2-P, respectively. Cultures were harvested for biomass which was lyophilized and subjected to FAMEs analysis to obtain oil profiles, shown in FIGS. 36 and 37.

Interestingly, different PUFA products accumulated in 173 transformation series. In one recovered auxotroph, 173-1, EPA accumulates at ˜5.0% in axenic strains in the flask. In the other recovered auxotroph from the same transformation, 173-2, DPA-3 accumulates at ˜4.0% in axenic strains in flask, shown in FIG. 35. Without wishing to be bound by any theory, an elongase in WT T18 that acts on EPA, converting it to DPA n-3, appears to be inactivated in 173-1.

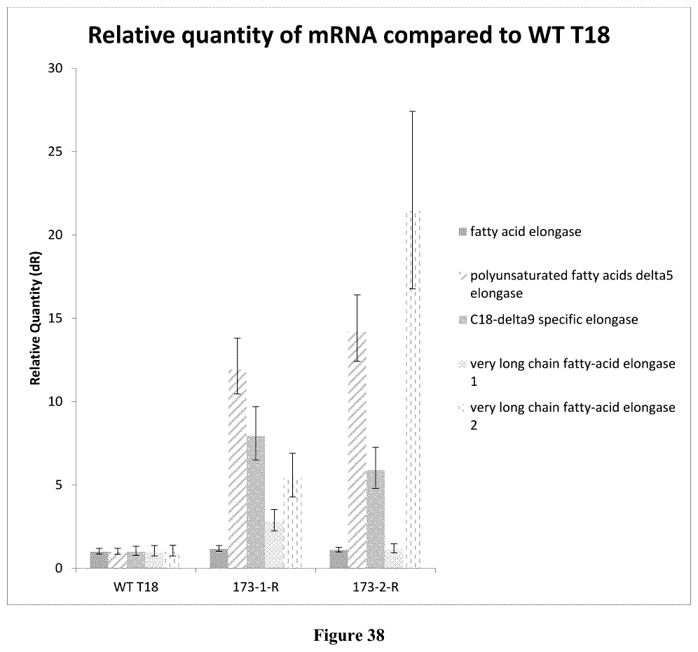

In an effort to determine the identity of this elongase, a RT-qPCR experiment was conducted with five prospective elongases chosen from annotated sequencing data. These elongases were called “fatty acid elongases” “polyunsaturated fatty acids 45 elongases,” “C18-Δ9 specific elongase,” “very long chain fatty-acid elongase 1,” and “very long chain fatty-acid elongase 2”. Results do not indicate a completely inactivated elongase in 173-1-R, but there is a candidate that seemingly experiences much greater up-regulation in 173-2-R: very long chain fatty-acid elongase 2. The best candidate native elongase for up-regulation in recovered auxotrophs, causing the conversion of g-linolenic acid to DGLA, is polyunsaturated fatty acids 45 elongase (FIG. 38).

### Example 18. Engineered Microorganisms Subjected to Adaptive Evolution

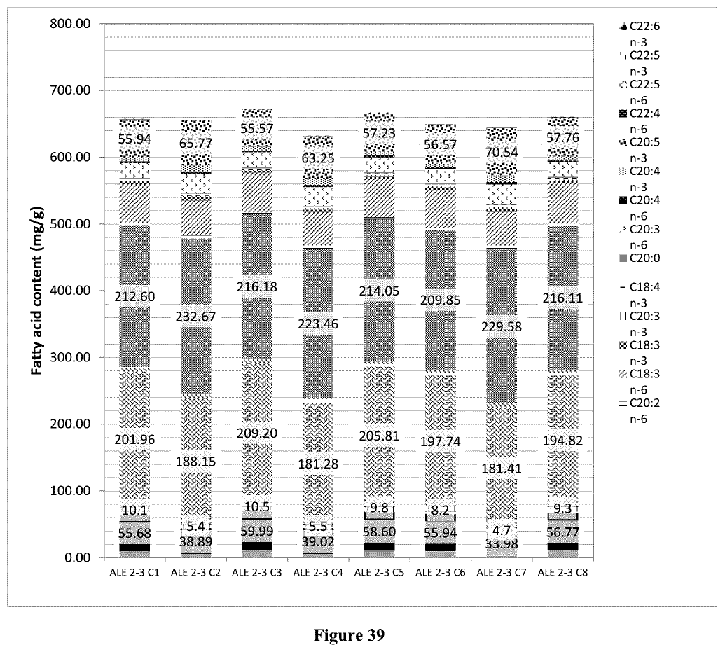

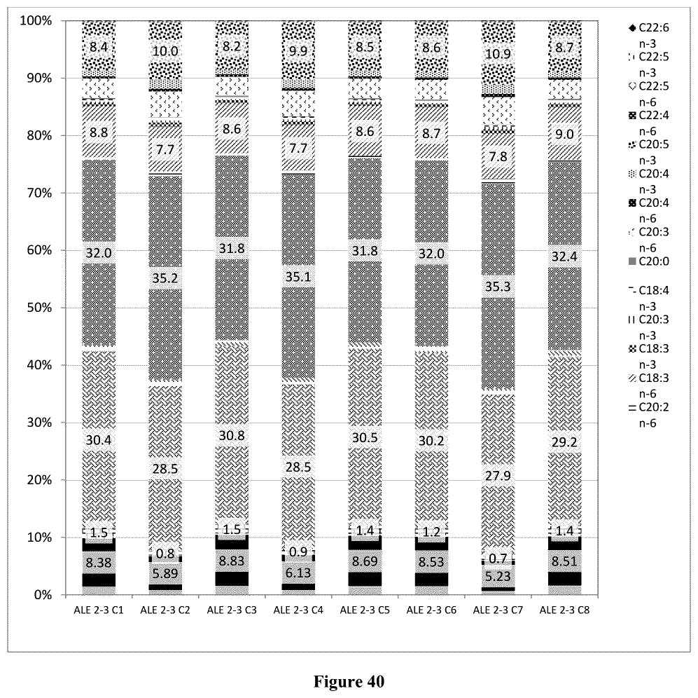

Transformant 173-1-R was subjected to Adaptive Laboratory Evolution. In this method, the transformant is passaged 30 times in 10 mL WDL+30 g/L NaCl. Each passaged culture grows for 24 hours at 25ºC, then at 4ºC for 48 hours, after which the culture is used to seed 10 mL of fresh 10 mL WDL+30 g/L NaCl and subjected to the same temperature cycle. After 30 rounds of passaging, single colonies were obtained from the resultant mixed population. The axenic transformants were grown in 60 mL UF60 medium at 20° ° C. for 10-14 days (until glucose depletion). The FAMEs profiles of seven axenic transformants isolated from the 173-1-R ALE mixed population are shown in FIGS. 39 and 40.

### Example 19. Engineered Microorganisms Subjected to Mutagenesis

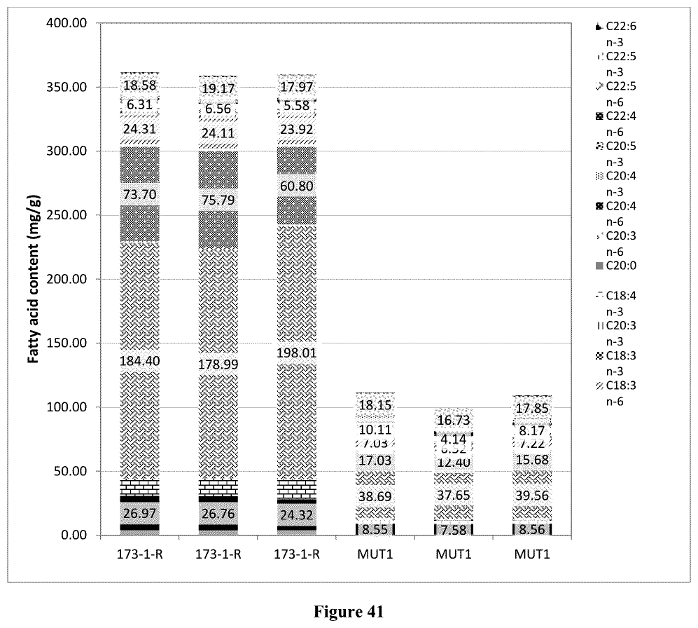

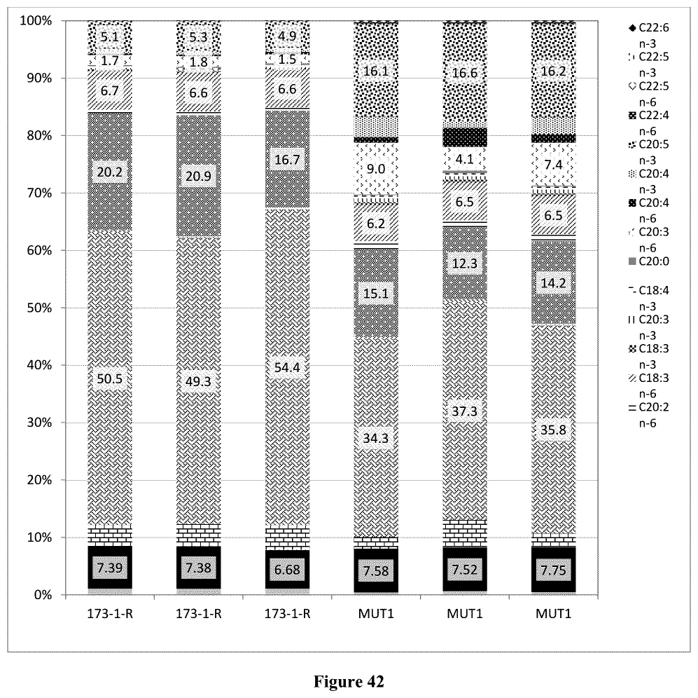

Transformant 173-1-R was subjected to mutagenesis in 10 mL WDL containing 0.3 M ethyl methanesulfonate (EMS). Working in the dark, the culture was incubated at 28° C., 200 rpm for 1 hour. The culture was pelleted at 1000g for 3 minutes, washed with phosphate-buffered saline (PBS), resuspended in 10 mL WDL and allowed to recover for 3 days in the dark at 25° C., 200 rpm. A 100 μL recovered culture was used to inoculate 10 mL WDL and allowed to grow in the dark at 25° C., 200 rpm, for 4 days. This culture was pelleted at 1000g, for 3 minutes and resuspended in 8 mL PBS. Four mL of resuspended cells were stained with 4,4-Difluoro-4-bora-3a,4a-diaza-s-indacene (BODIPY) (added 1 μL BODIPY and 397 μL 50% glycerol) and incubated at room temperature in the dark for 20 minutes. The cells were pelleted at 1000g, 3 minutes and washed with 4 mL PBS, then pelleted again and resuspended in 4 mL PBS. One milliliter (1 mL) of resuspended cells were added to 3 mL PBS and single cells were sorted by FACS with a Bio-Rad S3e Cell Sorter into a 96 well plate with WDL medium. After sorting, the plate was incubated in a BioTek Synergy H1 Hybrid Reader 25° C., 72 hours with shaking. One well showed significant growth and this strain was called 173-1-R MUT1. 173-1-R MUT1 was grown in 60 mL WDL, in triplicate, baffled flasks, at 25° C., and 200 rpm, for 6-7 days (until glucose depletion). FAMEs profiles for these transformants are shown in FIGS. 41 and 42.

### Example 20. Engineered Microorganisms Subjected to Mutagenesis

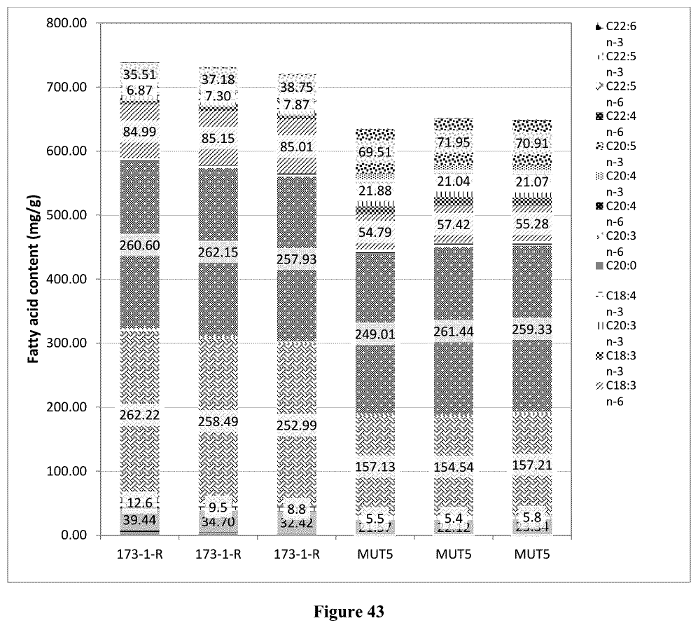

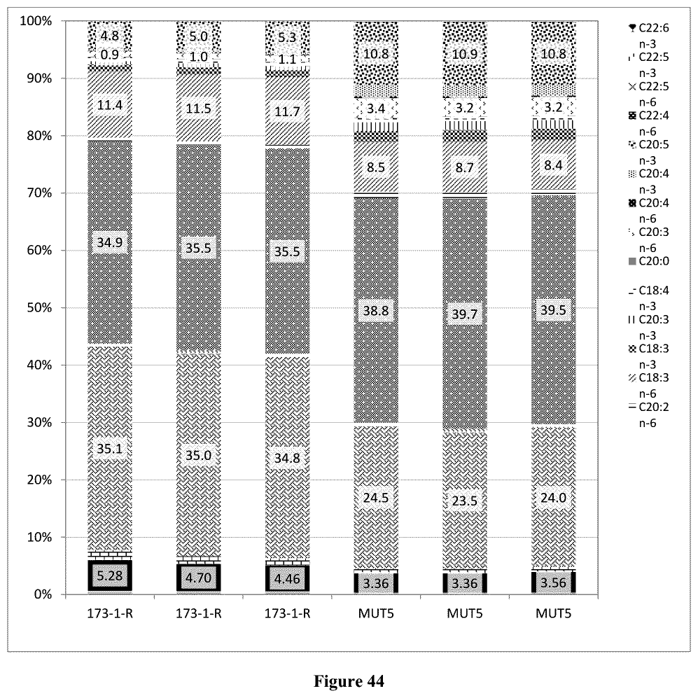

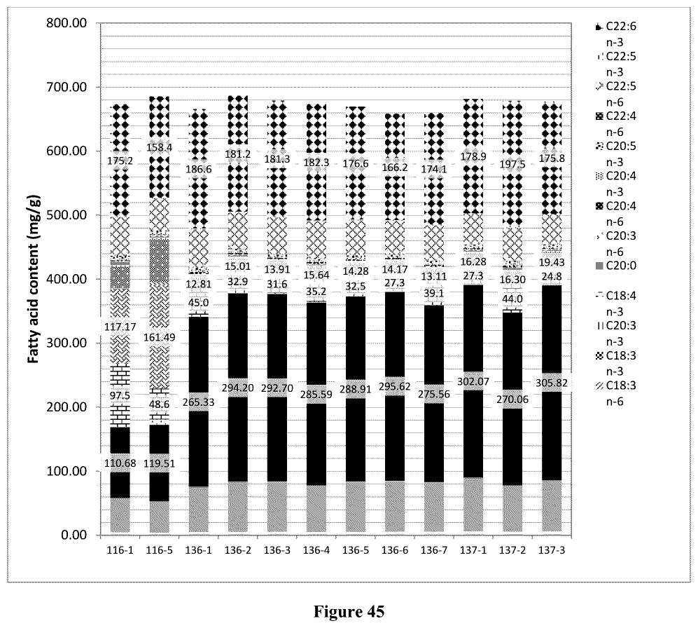

Transformant 173-1-R was subjected to mutagenesis in 10 mL WDL containing 0.35 M ethyl methanesulfonate (EMS). Working in the dark, the culture was incubated at 28° C., at 200 rpm for 1 hour. The culture was pelleted at 1000 g for 3 minutes, washed with PBS, resuspended in 10 mL WDL and allowed to recover for 7 days in the dark at 25° C., and at 200 rpm. 100 μL recovered culture was used to inoculate 10 mL WDL+1.5 mM isoniazid+12.5 uM cerulenin and allowed to grow for 3 days at 25° C., and at 200 rpm. Three milliliters of culture was pelleted at 1000g, for 3 minutes and resuspended in 3 mL PBS. Resuspended cells were stained with BODIPY (added 1.5 μL BODIPY and 6.5 μL DMSO) and incubated at room temperature in dark for 20 minutes. The cells were pelleted at 1000g, for 3 minutes and washed with 3 mL PBS, and then pelleted again and resuspended in 3 mL PBS. Single cells were sorted by FACS with a Bio-Rad S3e Cell Sorter into a 96 well plate with WDL medium. After sorting, the plate was incubated in a BioTek Synergy H1 Hybrid Reader 25C, 14 days with shaking. A transformant isolated from growth in one of the wells was designated 173-1-R MUT5. The 173-1-R and 173-1-R MUT5 strains were grown in 60 mL UF60, in triplicate, baffled flasks, at 20° C., and at 200 rpm, for 13 days (until glucose depletion). FAMEs profiles are shown in FIGS. 43 and 44. During the course of the above oil profile engineering attempts, it became apparent that there was a unique transcriptional activity conferred by the juxtapositioning of the native Δ9 desaturase promoter and the C16 elongase ORF, connected by a short junction sequence. This transcriptional activity was vastly diminished when Δ6 desaturase (Bty) or Ω-3 desaturase (Obl) was located 3′ to the Δ9 desaturase promoter. This effect may be seen in FIG. 45 comparing series 116 (C16 elo 3′ to Δ9 PR), series 136 (Δ6 des Bty 3′ to Δ9 PR) and series 137 Ω-3 desaturase Obl 3′ to A9 PR). There is about a 10-fold reduction in the production of oleic acid in 136 and 137 series compared to 116 series transformants. The reduced activity of C16 elongase (Obl) on C16:0 in 136 and 137 series results in accumulation of C16:0 to over 2.5 times amount in 116 series.

An unexpected and unintended effect of engineering the classical pathway in T18 was the increased production of carotenoids as well as the production of novel carotenoid species not generally seen in T18. When oil extracted from a fermentation of 173-1-R, a recovered auxotroph and EPA-producer, was analyzed by HPLC and compared to a sample of oil from WT T18, there was a 93% increase in B-carotene content and a 203% increase in canthaxanthin content.

### Example 21. Engineered Microorganisms Comprising the T18 Wild Type Polyunsaturated Fatty Acids Delta 5 Elongase (5ELO)

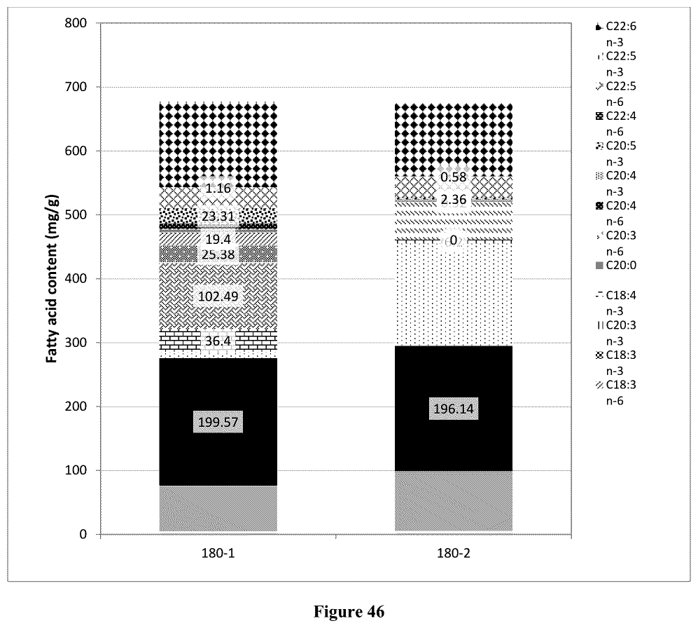

Based on qPCR results, D5 elongase, called 5ELO from WT T18 was cloned into an expression vector 3′ to C16:0 elongase (Obl) and Δ9 desaturase (Ulk), a zeocin resistance gene (ble^{R}), Δ6 desaturase (Bty) and 5′ to Ω-3 desaturase (Pav) (2 copies), Δ5 desaturase (Thr) and A12 desaturase (Thr) under the control of the native T18 Δ9 desaturase promoter and separated by 2A sequences resulting in pHR101, shown in FIG. 2. T18 WT was biolistically transformed with pHR101, resulting in zeocin-resistant transformants, referred to herein as the 180 series transformants. Fatty acid profiles of the 180 series transformants 180-1 and 180-2 grown in 10% N WDL to glucose depletion were determined by FAMEs analysis as shown in FIG. 46. T18 WT has been demonstrated to make very little EPA and no C18:2 linoleic acid, while 180-1 produces 23.3 mg/g EPA and 25.4 mg/g linoleic acid which represent 3.4% and 3.7% weight percent of the total fatty acids. Weight percent determined by mg/g specific fatty acid over mg/g total fatty acids. Strain 180-1 produced EPA at a level comparable to 173-1-R: 23.3 mg/g vs 24.6 mg/g respectively.

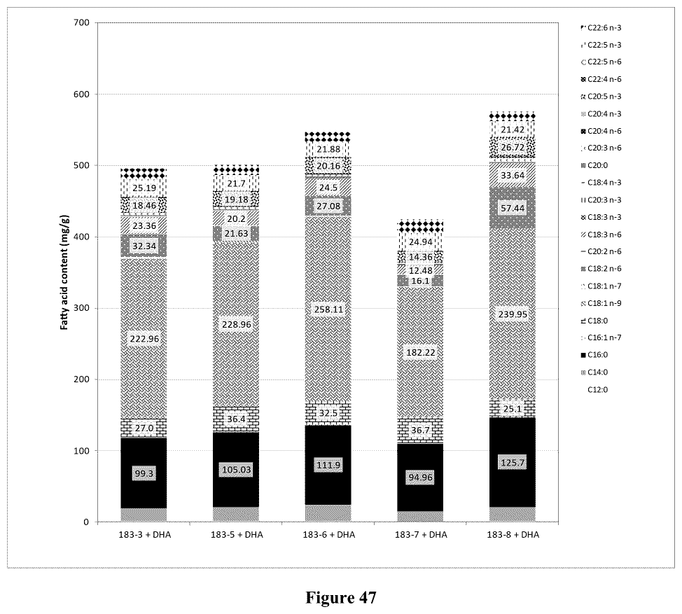

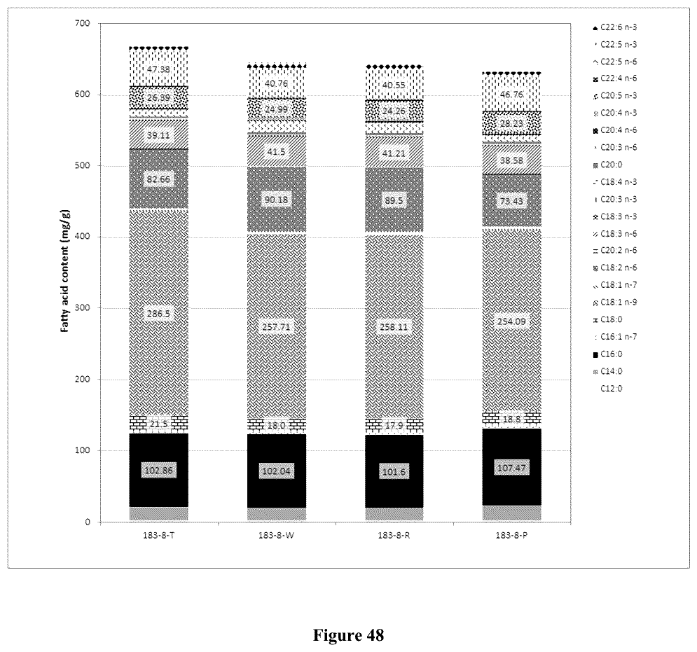

Strain 180-1 was biolistically transformed with pHR64 with the intention of knocking out subunit B of the PUFA synthase. G418-resistant transformants were obtained, referred to herein as the 183 series transformants. Fatty acid profiles of 183-3 and 183-(5-8) transformants grown in 10% N WDL+0.6 mM DHA until there was 18.7, 22.2, 14.2 or 29.3 g/L glucose remaining, respectively (no glucose reading for 183-8 due to YSI device error), were determined by FAMEs analysis as shown in FIG. 47. Strain 183-8 was recovered to prototrophy using methods described previously resulting in axenic strains 183-8-T, 183-8-W, 183-8-R and 183-8-P. Fatty acid profiles of 183-8-T,W,R and -P grown in 10% N WDL until there was 3.63, 3.33, 3.86 and 5.81 g/L glucose remaining, respectively, were determined by FAMEs analysis as shown in FIG. 48. The 183-8 prototrophs reached 47 mg/g DPA n-3.

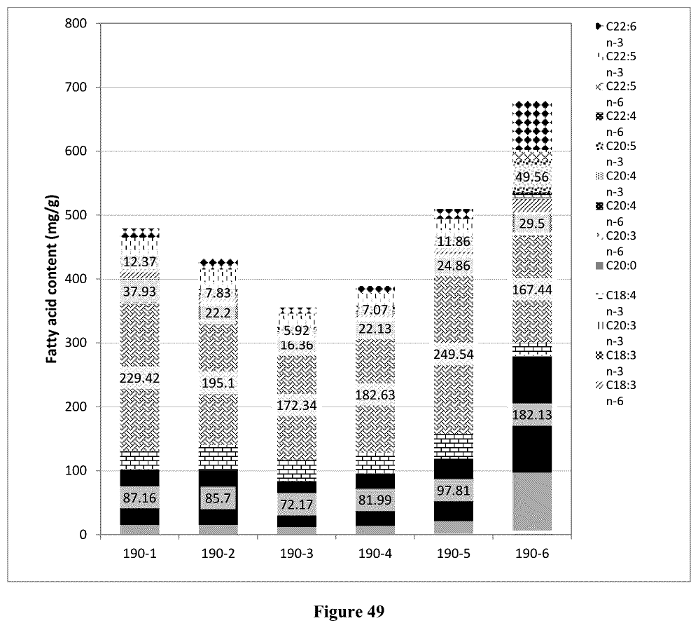

Strain 180-1 was biolistically transformed with pHR 106 with the intention of knocking out very long-chain fatty acids elongase 2, called VLCELO2. Hygromycin-resistant transformants were obtained, referred to herein as 185 series transformants. 185-6 was biolistically transformed with pHR95 with the intention of knocking out subunit B of the PUFA synthase. G418-resistant transformants were obtained, referred to herein as the 190 series transformants. Fatty acid profiles of 190-(1-6) transformants grown in 10% N WDL+0.6 mM DHA, except 190-6, grown in 10% N WDL, until there was 18.5, 28.6, 32.3, 29.0, 17.0 or 0 g/L glucose remaining, respectively, were determined by FAMEs analysis as shown in FIG. 49. Strain 190-6, despite the appearance of a working PUFA synthase, produced 49.56 mg/g EPA, more than double the proportion in 180-1.

1. A microbial oil comprising fatty acids, wherein the fatty acids comprise C20:3(n-6) (di-homo-γ-linoleic acid) and C20:5(n-3) eicosapentaenoic acid (EPA).

2. The microbial oil of claim 1, wherein the fatty acids comprise 0.01% to 16% C20:3(n-6) (di-homo-γ-linoleic acid).

3. The microbial oil of claim 1, wherein the fatty acids comprise 1-17% EPA.

4. The microbial oil of claim 3, wherein the fatty acids comprise 5-17% EPA.

5. The microbial oil of claim 1, wherein the fatty acids comprise 5-10% C14:0 (myristic acid).

6. The microbial oil of claim 1, wherein the fatty acids comprise 13-22% of C16:0 (hexadecanoic acid).

7. The microbial oil of claim 1, wherein the fatty acids comprise C18 unsaturated fatty acids.

8. The microbial oil of claim 7, wherein the fatty acids comprise 10-60% C18 unsaturated fatty acids.

9. The microbial oil of claim 7, wherein the fatty acids comprise 10-45% C18:1 oleic acid.

10. The microbial oil of claim 7, wherein the fatty acids comprise C18:2 (n-6) linoleic acid.

11. The microbial oil of claim 10, wherein the fatty acids comprise 0.01% to 40% linoleic acid.

12. The microbial oil of claim 1, wherein the oil further comprises C18:3(n-3) (α-linoleic acid), C18:3(n-6) (γ-linoleic acid), C20:4(n-3) (eicosatetraenoic acid), C20:5(n-3) (EPA), and C22:5(n-3) (docosapentaenoic acid (n-3) (DPA-3).

13. The microbial oil of claim 1, wherein the microbial oil comprises 85% to 95% total fatty acids by weight.

14. The microbial oil of claim 13, wherein the microbial oil comprises at least 90% total fatty acids by weight.

15. The microbial oil of claim 1, wherein the microbial oil comprises less than 35% or less than 30% saturated fatty acids.

16. (canceled)

17. The microbial oil of claim 1, wherein the microbial oil comprises 0.001% to 35% saturated fatty acids.

18. (canceled)

19. (canceled)

20. An engineered microorganism comprising a first nucleic acid sequence encoding an elongase and a second nucleic acid sequence encoding a desaturase, wherein the first and second nucleic acid sequences are operably linked to a delta 9 desaturase promoter.

21. The engineered microorganism of claim 20, wherein the desaturase is a delta 9 desaturase.

22. The engineered microorganism of claim 21, wherein the delta 9 desaturase is a Thraustochytrium sp. or Ulkenia sp. delta 9 desaturase.

23. The engineered microorganism of claim 20, wherein the elongase is a C16:0 elongase or a Delta 5 elongase.

24. The engineered microorganism of claim 23, wherein the C16:0 elongase is a Oblongichytrium sp. elongase.

25. The engineered microorganism of claim 20, further comprising a third nucleic acid encoding a delta 12 desaturase

26. The engineered microorganism of claim 25, wherein the delta 12 desaturase is a Thraustochytrium sp. desaturase.

27. The engineered microorganism of claim 20, further comprising a fourth nucleic acid encoding a delta 6 desaturase.

28. The engineered microorganism of claim 27, wherein the delta 6 desaturase is a Botryochytrium sp. delta 6 desaturase.

29. The engineered microorganism of claim 20, further comprising a fifth nucleic acid encoding an omega 3 desaturase.

30. The engineered microorganism of claim 29, wherein the omega 3 desaturase is a Oblongichytrium sp. omega 3 desaturase.

31. The engineered microorganism of claim 20, further comprising a sixth nucleic acid encoding a delta 5 desaturase.

32. The engineered microorganism of claim 31, wherein the delta 5 desaturase is a Thraustochytrium sp. delta 5 desaturase.

33. The engineered microorganism of claim 20, wherein the delta 9 desaturase promoter is located in its native position in the genome of the microorganism.

34. The engineered microorganism of claim 20, wherein the delta 9 desaturase promoter and first and second nucleic acids are located on a heterologous construct.

35. The engineered microorganism of claim 20, wherein the first and second nucleic acids disrupt the microorganism's endogenous delta 9 desaturase sequence.

36. The engineered microorganism of claim 20, further comprising a zeocin resistance gene.

37. The engineered microorganism of claim 20, further comprising one or more 2A sequences.

38. The engineered microorganism of claim 20, further comprising a reporter gene.

39. The engineered microorganism of claim 38, wherein the reporter gene is luciferase.

40. The engineered microorganism of claim 20, further comprising one or more tubulin promoters, one or more tubulin terminators, or both one or more tubulin promoters and one or more tubulin terminators.

41. The engineered microorganism of claim 20, wherein the nucleic acid comprises one or more PUFA synthase subunit B promoters, one or more PUFA synthase subunit B terminators, or both one or more PUFA synthase subunit B promoters and one or more PUFA synthase subunit B terminators.

42. A microbial oil produced by the engineered microorganism of claim 20.

43. A method for producing polyunsaturated fatty acids comprising
(a) providing the engineered microorganism of claim 20 and
(b) culturing the engineered microorganism under conditions sufficient to produce the polyunsaturated fatty acids.

44. A method of promoting conversion of saturated fatty acids to unsaturated fatty acids comprising the steps of
(a) transforming an oil-producing microorganism with a construct comprising a first nucleic acid encoding an elongase and a second nucleic acid encoding a desaturase, wherein the construct is inserted into the genome of the oil-producing microorganism at a location wherein expression of the encoded elongase and desaturase is controlled by a promoter native to the oil-producing microorganism; and
(iii) culturing the transformed microorganisms under conditions to produce fatty acids, wherein the transformed microorganisms convert saturated fatty acids to unsaturated fatty acids greater than a control untransformed microorganism.

45.-67. (canceled)
